(19) United States
(12) Patent Application Publication
(10) Pub. No.: US 2024/0215639 A1
(43) Pub. Date: Jul. 4, 2024

(54) INTRAORAL AEROSOL DELIVERY DEVICE

(71) Applicant: Oron ZACHAR, Tel Aviv (IL)

(72) Inventor: Oron ZACHAR, Tel Aviv (IL)

(21) Appl. No.: 18/379,681

(22) Filed: Oct. 13, 2023

Related U.S. Application Data
(60) Provisional application No. 63/417,871, filed on Oct. 20, 2022.

(51) Int. Cl. A24F 40/10; A24F 40/40; A61M 15/00; A61M 15/06

(52) U.S. Cl. CPC A24F 40/10; A24F 40/40; A61M 15/0001; A61M 15/06; A61M 2205/33

(57) ABSTRACT

An electrically-powered inhalation device for delivery of an aerosol to the oropharynx of a user includes a distal portion housing a piezo assembly including an ultrasonically vibrable mesh membrane, and a neck portion including a narrow section characterized by a minimum cross-sectional dimension that is at least 10% smaller than a minimum cross-sectional dimension of the distal portion passing through and parallel to the mesh membrane. At least a part of the narrow section is displaced proximally from the mesh membrane by at least 0.5 cm and not more than 6 cm. The inhalation device is shaped such that when the user's lips and/or teeth are transversely engaged with the narrow section, the mist-generating location resides distal to the user's teeth within the user's oral cavity and the mist-exiting location is in direct fluid communication with the user's oropharynx.

### CROSS-REFERENCE TO RELATED APPLICATIONS

The present application hereby includes by reference U.S. provisional 63/305,009 filed on 2022 Jan. 31, the disclosure of which is incorporated herein by reference.

### FIELD OF THE INVENTION

The present invention relates to mist-delivery devices and refillable and/or replaceable containers for use therein, and to methods for using such devices. In particular the present invention relates to devices for intraoral use for delivering an aerosol to a user's oropharynx.

### BACKGROUND

Existing oral inhalers suffer from the problem that any mist produced must traverse the tongue and other parts of the oral cavity, causing part of any dosed substance to fail to reach the oropharynx. Therefore, a need exists for an intraoral inhaler capable of delivering a precise dosage of a substance to a user's oropharynx, preferably configured to place a mist-generating location and/or mist-exiting location of the inhaler far enough into the oral cavity to overcome the aforementioned shortcoming. There is also a need for such an inhalation device to be compact and comfortable to use.

### SUMMARY

According to embodiments disclosed herein, an electrically-powered inhalation device for delivery of an aerosol to the oropharynx of a user comprises: (a) respective proximal and distal portions, the proximal portion including an inlet for a liquid, the distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the inhalation device is shaped such that when the user's lips and/or teeth are transversely engaged with the intermediate portion, the mist-generating location resides within the user's oral cavity and the mist-exiting location is in direct fluid communication with the user's oropharynx.

According to embodiments, an electrically-powered inhalation device for delivery of an aerosol to the oropharynx of a user comprises: (a) respective proximal and distal portions, the proximal portion including an inlet for a liquid, the distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the distal portion is dimensioned to vertically span the user's oral cavity from tongue to hard-palate when the user's lips and/or teeth are transversely engaged with the intermediate portion, so as to place the mist-exiting location in fluid communication with the user's oropharynx.

In some embodiments, the liquid-inlet can be configured to receive liquid from a container, the liquid-inlet and the container having respective mating arrangements for mating with each other. In some embodiments, the mating can be reversible.

In some embodiments, the container can be detachably attachable to the proximal portion. In some embodiments, the inhalation device can additionally comprise the container. In some embodiments, the proximal portion can comprise a compartment for storing the liquid.

In some embodiments, the inhalation device can additionally comprise a portable power source. In some embodiments, the inhalation device can additionally comprise an inhalation sensor for monitoring a flow in an inhalation flow-path. In some embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation-path.

In some embodiments, the inhalation device can additionally comprise an exhalation sensor for monitoring a flow in an exhalation-flow path. In some embodiments, the exhalation sensor can be configured to detect a concentration of a chemical compound in the exhalation-flow path. In some embodiments, the chemical compound can be a component of the liquid. In some embodiments, the inhalation device can comprise control circuitry configured to cease or delay activation of the mesh membrane in response to a result of the monitoring of the flow in the exhalation flow path.

In some embodiments, the mesh membrane can be effective to eject at least 5 times, or at least 10 times, or at least 20 times, or at least 50 times more liquid in the mist during user inhalation than during user exhalation.

In some embodiments, the inhalation device can comprise an inhalation flow-path and an exhalation flow-path, each of the flow-paths including a respective one-way fluid valve.

In some embodiments, at least a portion of the distal portion can comprise a coating for generating a taste and/or odor sensation. In some embodiments, at least a portion of the intermediate portion can comprise a coating for generating a taste and/or odor sensation.

In some embodiments, the inhalation device comprises control circuitry programmable to cause the mesh membrane to eject, in the mist, a liquid quantity that is either predetermined or received in an input from a user.

In some embodiments, at least a portion of the container can be above a plane longitudinally bisecting the intermediate portion when the device is rotated such that the plane is horizontal. In some embodiments, all of the container can be above a plane longitudinally bisecting the intermediate portion when the device is rotated such that the plane is horizontal.

In some embodiments, the distal portion can comprise a liquid-retaining compartment in fluid communication with the liquid inlet via a conduit, and the liquid-retaining compartment can be shaped to receive a quantity of the liquid via the conduit by force of gravity when the inhalation device is in a first orientation, and to retain at least a part of the quantity against the force of gravity when the inhalation device is in a second orientation. In some embodiments, the retaining can be by a wall of the liquid-retaining compartment, and wall can be effective to partially block an egress of the retained at least a part of the quantity.

In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, a maximum retainable fluid capacity of the liquid-retaining compartment is at least 0.5 cc and not more than 4 cc, or at least 1 cc and not more 3 cc, or at least 1.5 cc and not more 2.5 cc.

In some embodiments, a ratio of (i) a combined fluid capacity of the container and the conduit to (ii) a maximum retainable fluid capacity of the liquid-retaining compartment, can be at least 1 and not more than 4, or at least 1.5 and not more than 3, or at least 1.75 and not more than 2.5.

In some embodiments, the inhalation device can additionally comprise a capillary pathway for conveying a portion of the liquid by capillary action from the liquid-inlet to the mesh membrane or to within 1 mm of the mesh membrane.

In some embodiments, the mist-generating location can be at least 20% deep or at least 30% deep or at least 40% deep or at least 50% deep or at least 60% deep or at least 70% deep or at least 80% deep into an oral-cavity volume beneath the user's hard palate.

In some embodiments, the inhalation device can additionally comprise a display device configured to display information about at least one of: (i) a currently-remaining quantity of the liquid or of a component thereof, (ii) an already-misted quantity of the liquid or of a component thereof, and/or (iii) the identity of a component of the liquid.

In some embodiments in which the inhalation device includes an exhalation sensor, the inhalation device can additionally comprising a display device configured to display information about at least one of: (i) a currently-remaining quantity of the liquid or of a component thereof, (ii) an already-misted quantity of the liquid or of a component thereof, (iii) the identity of a component of the liquid, and (iv) the detected concentration of the chemical compound in the exhalation-flow path.

According to embodiments disclosed herein, an electrically-powered inhalation device for delivery of an aerosol to the oropharynx of a user comprises: (a) respective proximal and distal portions, the distal portion including (i) a volume for storing a liquid, (ii) an aerosol outlet defining a mist-exiting location and (iii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the inhalation device is shaped such that when the user's lips and/or teeth are transversely engaged with the intermediate portion, the mist-generating location resides within the user's oral cavity and the mist-exiting location is in direct fluid communication with the user's oropharynx.

According to embodiments, an electrically-powered inhalation device for delivery of an aerosol to the oropharynx of a user comprises: (a) respective proximal and distal portions, the distal portion including (i) a volume for storing a liquid, (ii) an aerosol outlet defining a mist-exiting location and (iii) a piezo assembly including an ultrasonically vibrable mesh membrane, for producing, upon electrical activation, a mist comprising droplets of the liquid, the mesh membrane defining a mist-generating location; and (b) an intermediate portion disposed distally from the proximal portion and proximally from the distal portion, wherein the distal portion is dimensioned to vertically span the user's oral cavity from tongue to hard-palate when the user's lips and/or teeth are transversely engaged with the intermediate portion, so as to place the mist-exiting location in fluid communication with the user's oropharynx.

In some embodiments, the inhalation device can additionally comprise a portable power source. In some embodiments, the inhalation device can additionally comprise an inhalation sensor for monitoring a flow in an inhalation flow-path. In some embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation-path.

In some embodiments, the inhalation device can additionally comprise an exhalation sensor for monitoring a flow in an exhalation-flow path. In some embodiments, the exhalation sensor can be configured to detect a concentration of a chemical compound in the exhalation-flow path. In some embodiments, the chemical compound can be a component of the liquid. In some embodiments, the inhalation device can comprise control circuitry configured to cease or delay activation of the mesh membrane in response to a result of the monitoring of the flow in the exhalation flow path.

In some embodiments, the mesh membrane can be effective to eject at least 5 times, or at least 10 times, or at least 20 times, or at least 50 times more liquid in the mist during user inhalation than during user exhalation. In some embodiments, the inhalation device can comprise an inhalation flow-path and an exhalation flow-path, each of the flow-paths including a respective one-way fluid valve.

In some embodiments, at least a portion of the distal portion can comprise a coating for generating a taste and/or odor sensation. In some embodiments, at least a portion of the intermediate portion can comprise a coating for generating a taste and/or odor sensation.

In some embodiments, the inhalation device comprises control circuitry programmable to cause the mesh membrane to eject, in the mist, a liquid quantity that is either predetermined or received in an input from a user.

In some embodiments, a maximum retainable fluid capacity of the liquid-retaining compartment is at least 0.5 cc and not more than 4 cc, or at least 1 cc and not more 3 cc, or at least 1.5 cc and not more 2.5 cc.

In some embodiments, a ratio of (i) a combined fluid capacity of the container and the conduit to (ii) a maximum retainable fluid capacity of the liquid-retaining compartment, can be at least 1 and not more than 4, or at least 1.5 and not more than 3, or at least 1.75 and not more than 2.5.

In some embodiments, the inhalation device can additionally comprise a capillary pathway for conveying a portion of the liquid by capillary action from the liquid-inlet to the mesh membrane or to within 1 mm of the mesh membrane.

In some embodiments, the inhalation device can additionally comprise a capillary pathway for conveying a portion of the liquid by capillary action from within the liquid-storing volume to the mesh membrane

In some embodiments, the mist-generating location can be at least 20% deep or at least 30% deep or at least 40% deep or at least 50% deep or at least 60% deep or at least 70% deep or at least 80% deep into an oral-cavity volume beneath the user's hard palate.

In some embodiments, the inhalation device can additionally comprise a display device configured to display information about at least one of: (i) a currently-remaining quantity of the liquid or of a component thereof, (ii) an already-misted quantity of the liquid or of a component thereof, and/or (iii) the identity of a component of the liquid.

In some embodiments in which the inhalation device includes an exhalation sensor, the inhalation device can additionally comprise a display device configured to display information about at least one of: (i) a currently-remaining quantity of the liquid or of a component thereof, (ii) an already-misted quantity of the liquid or of a component thereof, (iii) the identity of a component of the liquid, and (iv) the detected concentration of the chemical compound in the exhalation-flow path.

According to embodiments disclosed herein, an electrically-powered inhalation device for delivery of an aerosol to the oropharynx of a user comprises: (a) a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) a neck portion including a narrow section, the narrow section being characterized by a minimum cross-sectional dimension that is at least 10% smaller than a minimum cross-sectional dimension of the distal portion passing through and parallel to the mesh membrane, at least a part of the narrow section being displaced proximally from the mesh membrane by at least 0.5 cm and not more than 6 cm, wherein the inhalation device is shaped such that when the user's lips and/or teeth are transversely engaged with the narrow section, the mist-generating location resides distal to the user's teeth within the user's oral cavity and the mist-exiting location is in direct fluid communication with the user's oropharynx.

In some embodiments, the distal portion can comprise a distal casing encompassing the mesh membrane at least circumferentially.

In some embodiments, the at least a part of the narrow section can be displaced proximally from the mesh membrane by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

In some embodiments, the narrow section can be characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

In some embodiments, the minimum cross-sectional dimension of the narrow section and the minimum cross-sectional dimension of the distal portion can define vectors that are coplanar, or within ±15° of being coplanar, or within ±30° of being coplanar, or within ±45° of being coplanar.

In some embodiments, the inhalation device of can comprise a proximal portion that includes a power source for powering the piezo assembly.

In some embodiments, the inhalation device can comprise a proximal portion that includes a liquid inlet.

In some embodiments, the inhalation device can comprise a first proximal portion that includes a liquid inlet and a second proximal portion that includes a power source for powering the piezo assembly.

In some embodiments, it can be that an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

In some embodiments, a center of gravity of the inhalation device can be is displaced proximally from a distal end of the narrow section when the inhalation device is in a liquid-empty state.

In some embodiments, the inhalation device additionally comprising an inhalation sensor for monitoring a flow in an inhalation flow-path. In some such embodiments, the inhalation sensor can be effective to detect an air pressure in the inhalation-flow path. In some embodiments, the inhalation sensor can be effective to detect a difference between an air pressure in the inhalation flow-path and an ambient air pressure outside the inhalation device. In some embodiments, the inhalation device can comprise control circuitry configured to initiate and/or cease activation of the mesh membrane in response to a result of the monitoring of the flow in the inhalation flow path.

In some embodiments, the distal portion can comprise a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation. In some such embodiments, the retaining can be by a wall of the liquid-retaining compartment, the wall being effective to partially block an egress of the retained at least a part of the quantity. In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, the inhalation device can be shaped such that when the user's lips and/or teeth are transversely engaged with the intermediate portion, the mist-generating location is at least 20% deep, or at least 30% deep, or at least 40% deep, or at least 50% deep, or at least 60% deep, or at least 70% deep, or at least 80% deep, into an oral-cavity volume beneath the user's hard palate.

In some embodiments, a kit can comprise the inhalation device according to any of the embodiments disclosed hereinabove, packaged in a container such that the proximal portion that includes a liquid inlet is detached from the neck portion.

According to embodiments disclosed herein, an electrically-powered inhalation device for delivery of an aerosol to the oropharynx of a user comprises: (a) a distal portion including (i) an aerosol outlet defining a mist-exiting location and (ii) a piezo assembly including an ultrasonically vibrable mesh membrane, for producing, upon electrical activation, a mist comprising droplets of the liquid, the mesh membrane defining a mist-generating location; and (b) a neck portion including a narrow section, the narrow section being characterized by a minimum cross-sectional dimension that is at least 10% smaller than a minimum cross-sectional dimension passing through and parallel to the mesh membrane, a center of gravity of the inhalation device being displaced proximally from a distal end of the narrow section when the inhalation device is in a liquid-empty state, wherein the inhalation device is shaped such that when the user's lips and/or teeth are transversely engaged with the narrow section, the mist-generating location resides within the user's oral cavity and the mist-exiting location is in direct fluid communication with the user's oropharynx.

In some embodiments, the distal portion can comprise a distal casing encompassing the mesh membrane at least circumferentially.

In some embodiments, the narrow section can be characterized by a minimum cross-sectional dimension that is at least 20% smaller than the minimum cross-sectional dimension of the distal portion passing through and parallel to the mesh membrane, or at least 30% smaller than the minimum cross-sectional dimension of the distal portion passing through and parallel to the mesh membrane, or at least 40% smaller than the minimum cross-sectional dimension of the distal portion passing through and parallel to the mesh membrane, or at least 50% smaller than the minimum cross-sectional dimension of the distal portion passing through and parallel to the mesh membrane.

In some embodiments, the minimum cross-sectional dimension of the narrow section and the minimum cross-sectional dimension of the distal portion can define vectors that are coplanar, or within ±15° of being coplanar, or within ±30° of being coplanar, or within ±45° of being coplanar.

In some embodiments, the inhalation device can comprise a proximal portion that includes a power source for powering the piezo assembly.

In some embodiments, the inhalation device can comprise a proximal portion that includes a liquid inlet.

In some embodiments, the inhalation device can comprise a first proximal portion that includes a liquid inlet and a second proximal portion that includes a power source for powering the piezo assembly. In some embodiments, it can be that an outlet of the proximal portion that includes a liquid inlet is detachably attachable to the neck portion such that an interior volume of the proximal portion that includes a liquid inlet is arranged to be in fluid communication with an interior volume of the neck portion when a pressure-activated one-way valve is activated by pressure from the proximal portion that includes a liquid inlet.

In some embodiments, at least a part of the narrow section can be displaced proximally from the mesh membrane by at least 0.5 cm and not more than 6 cm, or by at least 0.5 cm and not more than 5.5 cm, or by at least 0.5 cm and not more than 5 cm, or by at least 0.5 cm and not more than 4.5 cm, or by at least 0.5 cm and not more than 4 cm, or by at least 1 cm and not more than 6 cm, or by at least 1 cm and not more than 5.5 cm, or by at least 1 cm and not more than 5 cm, or by at least 1 cm and not more than 4.5 cm, or by at least 1 cm and not more than 4 cm.

In some embodiments, the inhalation device can additionally comprise an inhalation sensor for monitoring a flow in an inhalation flow-path. In some such embodiments, the inhalation sensor can be effective to detect an air pressure in the inhalation-flow path.

In some embodiments, the inhalation sensor can be effective to detect a difference between an air pressure in the inhalation flow-path and an ambient air pressure outside the inhalation device.

In some embodiments, the inhalation device can comprise control circuitry configured to initiate and/or cease activation of the mesh membrane in response to a result of the monitoring of the flow in the inhalation flow path.

In some embodiments, the distal portion can comprise a liquid-retaining compartment in fluid communication with the neck portion, the liquid-retaining compartment being shaped to receive a quantity of the liquid from the neck portion by force of gravity when the inhalation device is in a first orientation, and to retain at least a part of the quantity against the force of gravity when the inhalation device is in a second orientation. In some such embodiments, the retaining can be by a wall of the liquid-retaining compartment, the wall being effective to partially block an egress of the retained at least a part of the quantity. In some embodiments, the second orientation can be such that substantially all of the mesh membrane is in liquid communication with the retained at least a part of the quantity. In some embodiments, the second orientation can be such that a surface liquid level in the liquid-retaining compartment is higher than a surface liquid level in the container.

In some embodiments, the inhalation device can be shaped such that when the user's lips and/or teeth are transversely engaged with the intermediate portion, the mist-generating location is at least 20% deep or at least 30% deep or at least 40% deep or at least 50% deep or at least 60% deep or at least 70% deep or at least 80% deep into an oral-cavity volume beneath the user's hard palate.

In some embodiments, a kit can comprise the inhalation device according to any of the embodiments disclosed hereinabove, packaged in a container such that the proximal portion that includes a liquid inlet is detached from the neck portion.

### BRIEF DESCRIPTION OF THE DRAWINGS

FIG. 1A is a schematic elevation drawing of an inhalation device, according to embodiments of the present invention.

FIG. 1B shows the inhalation device of FIG. 1A together with optional wick and removable liquid container, according to embodiments of the present invention.

FIG. 1C shows the inhalation device of FIG. 1B, with optional removal liquid container mated thereto, according to embodiments of the present invention.

FIG. 2A shows the inhalation device of FIG. 1C, in situ, in an activated state producing a mist in a user's oral cavity, according to embodiments of the present invention.

FIG. 2B schematically illustrates percentages of deepness into the volume beneath the hard palate.

FIG. 3 is a schematic elevation drawing of an inhalation device having a compact design, according to embodiments of the present invention.

FIG. 4 shows the inhalation device of FIG. 3, in situ, in an activated state producing a mist in a user's oral cavity, according to embodiments of the present invention.

FIG. 5 shows an inhalation device having inhalation and exhalation conveyances, in situ, in an activated state producing a mist in a user's oral cavity, according to embodiments of the present invention.

FIG. 6A and FIG. 6B and FIG. 6C show schematic views of an inhalation device according to embodiments of the present invention.

FIG. 7A and FIG. 7B show schematic views of an inhalation device according to embodiments of the present invention.

FIG. 8 shows an inhalation device according to embodiments of the present invention.

FIG. 9A and FIG. 9B and FIG. 9C and FIG. 9D are schematic cross-sectional illustrations of the inhalation device of FIG. 8 and a liquid, according to embodiments of the present invention.

FIG. 10A and FIG. 10B are cross-sectional views of inhalation devices according to embodiments of the present invention, showing liquid conduits having, respectively, circular and oval cross-sections.

FIG. 10C is a partial cutaway view of the proximal end of an inhalation device according to embodiments of the present invention, showing inhalation and exhalation sensors.

FIG. 11A and FIG. 11B are schematic cross-sectional illustrations of an inhalation device having a distal liquid-storage volume, according to embodiments of the present invention, at two respective orientations.

FIG. 12A and FIG. 12B are, respectively, schematic top- and side-view illustrations of an inhalation device having a display screen affixed to an intermediate portion of the inhalation device, according to embodiments of the present invention.

FIG. 13A and FIG. 13B are, respectively, schematic top- and side-view illustrations of an inhalation device having a display screen affixed to a proximal portion of the inhalation device, according to embodiments of the present invention.

FIG. 14 is an annotated schematic cross-sectional illustration of the inhalation device of FIGS. 9A-D.

FIG. 15A and FIG. 15B show schematic views of an inhalation device according to embodiments of the present invention, respectively assembled and unassembled, according to embodiments of the present invention.

FIG. 16 is an annotated schematic cross-sectional illustration of the inhalation device of FIGS. 15A-15B.

FIG. 17A and FIG. 17B are schematic illustrations of kits including inhalation devices, according to embodiments of the present invention.

FIG. 18A and FIG. 18B are schematic illustrations of kits including inhalation devices, according to embodiments of the present invention.

FIG. 19 illustrates a placement of the device in use by a human user, according to embodiments of the invention.

FIG. 20A and FIG. 20B are schematic illustrations of section cuts through the device to show some of the inside components and build elements.

FIG. 21A and FIG. 21B are schematic cross-sectional illustrations of an inhalation device having a distal liquid-storage volume, according to embodiments of the present invention, at two respective orientations.

FIG. 22A and FIG. 22B are schematic illustrations of kits including inhalation devices, according to embodiments of the present invention, including an assembly of a fluids compartment.

FIG. 23A and FIG. 23B and FIG. 23C and FIG. 23D are schematic illustrations of kits including inhalation devices, according to embodiments of the present invention, including an assembly of a fluids compartment.

FIG. 24A and FIG. 24B are schematic illustrations of kits including inhalation devices, according to embodiments of the present invention, including a safety element.

FIG. 25A and FIG. 25B and FIG. 25C and FIG. 25D are schematic illustrations of kits including inhalation devices, according to embodiments of the present invention, including an assembly with a soft vial or ampule insert, according to embodiments of the invention.

FIG. 26A and FIG. 26B illustrate an embodiment of the system with a prefilled liquid, according to embodiments of the invention.

FIG. 27A and FIG. 27B illustrate an embodiment of the system with a distal protection cover, according to embodiments of the invention.

FIG. 28A and FIG. 28B are schematic illustrations of an inhalation system, respectively in unassembled and assembles states, according to embodiments of the invention.

FIG. 29A and FIG. 29B are schematic illustrations of an inhalation system comprising a distal cap cover according to embodiments of the invention.

FIG. 30 is schematic illustrations of an inhalation system comprising a distal elastic band wrapping around a section of the delivery module according to embodiments of the invention.

FIG. 31 is schematic illustrations of an inhalation system comprising a bubble deflection mechanism.

FIG. 32A and FIG. 32B are schematic illustrations of an inhalation system comprising an air channel according to embodiments of the invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements.

Following is a list of reference numbers used in the figures for physiological features:
10—oral cavity
15—lips
20—teeth
25—tongue
30—hard palate
40—nasal cavity
50—oropharynx
60—velo-pharyngeal port
70—pharyngeal cavity

Note: Throughout this disclosure, subscripted reference numbers (e.g., 10₁ or 10_{A}) may be used to designate multiple separate appearances of elements of a single species, whether in a drawing or not; for example: 10₁ is a single appearance (out of a plurality of appearances) of element 10. The same elements can alternatively be referred to without subscript (e.g., 10 and not 10₁) when not referring to a specific one of the multiple separate appearances, i.e., to the species in general.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage. Physiological terms as used herein are to be understood according to their generally accepted meanings.

The terms ‘aerosol’ and ‘mist’ as used herein are synonymous and are used to describe a suspension of liquid droplets in air. The terms ‘inhalation device’ and ‘inhaler’ as used herein are synonymous and are used to describe a device that delivers an aerosol to a user's oral cavity.

The colloquial expression “smaller than”, e.g., in the context of “10% smaller than,” “20% smaller than” should be understood “smaller than by 10%,” smaller than by 20%,” etc., meaning, respectively, “90% as large,” “80% as large,” etc. Similarly, “at least 20% smaller” means “no more than 80% as large”.

An inhalation device is disclosed herein for delivering an aerosol of a liquid well inside the user's oral cavity such that the device largely prevents the user's tongue from interfering with the delivery of the aerosol to the user's oropharynx. The exemplary devices disclosed herein use a piezo assembly that includes an ultrasonically vibrable mesh membrane to generate the aerosol, and so the piezo assembly has an aerosol outlet that in intended use will release the aerosol where desired.

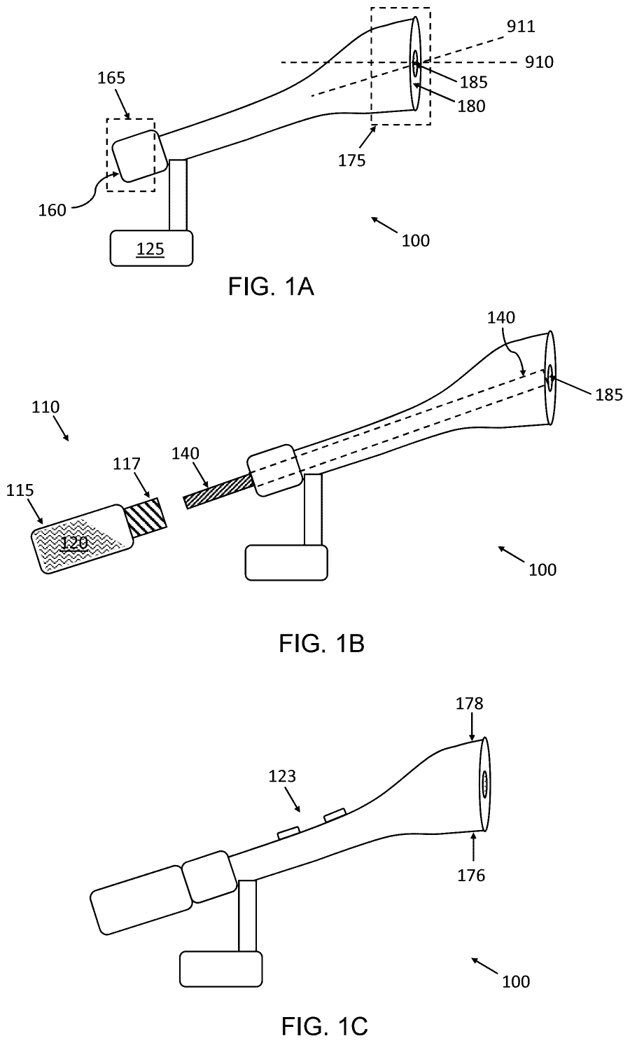

Referring now to the figures and in particular to FIGS. 1A, 1B and 1C, an inhalation device 100 according to embodiments is illustrated schematically. As seen in FIG. 1A, an inhalation device 100 has a distal portion 175 which includes the distal end of the inhalation device 100. The term ‘distal end’ is used herein to mean the end of the inhaler 100 at which an aerosol exits the inhaler 100. During normal intended use, the distal end is farthest from a user's hand, and/or is the first part of the device that enters a user's oral cavity. The term ‘distal’ may also used herein to indicate a direction towards the distal end. ‘Proximal’ as used herein refers to the end or direction which is opposite to the distal end or direction. The device 100 also includes a proximal portion 165. It should be noted that in some contexts the terms proximal portion and distal portion may be understood more broadly than the specific respective portions demarcated in FIG. 1A, and can refer to any portion that includes the respective end of the device.

FIGS. 1A-C show side elevation views, such that according to embodiments, the ‘top’ of the device 100 in each of the figures is the intended ‘top’ of the device 100 in actual use. Thus, in embodiments, an upper surface 178 of the distal portion 175 is intended to be ‘on top’ during use, and the lower surface 176 is intended to be ‘on the bottom’ during use. Nonetheless, in some embodiments, the inhalation device 100 is usable in other positions, e.g., with top and bottom reversed. The shape of the device 100 throughout the figures is shown as asymmetrical, i.e., the top of the device has a different contour than the bottom of the device. This can be beneficial for conforming to a user's oral cavity. Nonetheless, in some embodiments, the shape is symmetrical and does not have different contours on the top and the bottom of the device 100. In addition, the ‘thickness’ (dimension from top to bottom) of the distal portion 175 is shown as substantially thicker than the thickness of some of, most of, or all of the proximal portion 165. In embodiments, the distal portion 175 can have a thickness (e.g., maximum thickness, minimum thickness or average thickness) at least 30% or at least 50% or at least 100% greater than a corresponding thickness (respectively, maximum thickness, minimum thickness or average thickness) of the proximal portion 165. The relative thickness of the distal portion 175 can be useful in encouraging the user to place the distal portion 175 on top of the tongue so as to allow a mist generated by the device to directly reach the oropharynx.

The distal portion 175 includes a piezo assembly 180 that includes an ultrasonically vibrable mesh membrane 185. The mesh membrane 185 is the location at which an aerosol is generated/produced. In some embodiments (not shown) the distal portion can include an aerosol outlet displaced distally from the mesh membrane 185, where the aerosol exits the device 100 via such an aerosol outlet. This can be useful, for example for bringing the aerosol closer to the user's oropharynx or for directing the generated mist in a specific direction. Thus, in some embodiments the ‘mist-generating’ location and the ‘mist-exiting’ location are the same location (for example, in FIGS. 1A-1C) and in some embodiments these two locations are displaced from each other.

In embodiments, the proximal portion 165 can include a liquid inlet 160, through which a liquid 120 can be introduced into the device 100 for producing the mist. The liquid inlet is preferably mateable with a source of the liquid 120. In the example shown in FIG. 1B, the source of liquid is a replaceable/removable (i.e., attachable/detachable) container 110. The term ‘mateable’ is used herein to indicate that a mating arrangement exists, e.g., corresponding threading, snap-closures or appropriately sized inlet-outlet diameters. According to the example, the container 110 has a liquid storage volume 115 and an outlet 117. The outlet 117 is mateable with the liquid-inlet 160 of the device 100. FIG. 1B also shows a capillary pathway 140 for distally transporting liquid 120 in the direction of the mesh membrane 185. The capillary pathway 140 is typically disposed, and optionally held, so that a distal portion thereof is in contact with the mesh membrane 185, or displaced no more than 2 mm or no more than 1 mm from the mesh membrane 185. A proximal portion of the capillary pathway 140 is generally disposed within the liquid-storage volume 115 of the container 110 so as to establish a pathway for water transport from the liquid-storage volume 115 to the mesh membrane 85. In some embodiments, the container 110 is formed and/or provided as a component of the inhalation device 100.

A ‘capillary pathway’ 140 as the term is used herein is a material suitable for transport of a liquid) along a pathway by capillary action. Such a material often includes fibers, such as plant-based fibers e.g., cellulose, polymer-based fibers e.g., polyester, glass fibers e.g., in a woven fabric or bundled or unbundled glass fibers, or carbon fibers. In some non-limiting examples, the fibers can be very small, i.e., having diameters in the range of several or tens of microns. In other examples, the fibers can be larger. While the term “pathway” may appear to imply that a pathway for liquid transport to a mesh membrane may be a direct path, that is not necessarily the case. The transport of a liquid through the capillary pathway may include progression in random directions or omnidirectional progression. In some embodiments, the capillary pathway 140 can include fibers arranged so as to form direct pathways from various parts of the liquid-storage volume 115 but this is not necessary for the capillary transport to be effective. In some embodiments, the capillary pathway can comprise a hydrophilic material that is effective to facilitate transport of an aqueous liquid.

As shown in FIG. 1A, an inhalation device 100 according to embodiments can include comfort element(s) 123 for ease of placement of teeth and/or lips. Whenever ‘teeth’ are referred to herein in the context of placement of distance therefrom, then ‘teeth’ can be understood as ‘front teeth’. The two bumps shown in FIG. 1C are just one non-limiting example of such comfort elements; other non-limiting examples include depressions and single bumps. Such elements are not present in all designs within the scope of the present invention, but can be useful in some cases for optimal positioning of the device 100, and especially positioning of the distal portion 175, within the oral cavity. As shown in FIG. 1A. an inhalation device 100 according to embodiments can include a power and electronics module 125, which can include, for example, a power source (e.g., a battery or connection for mains electricity), wireless communication arrangements, and/or control circuitry. The control circuitry can include electronic hardware such as a printed circuit board, and firmware or software for operation of the device.

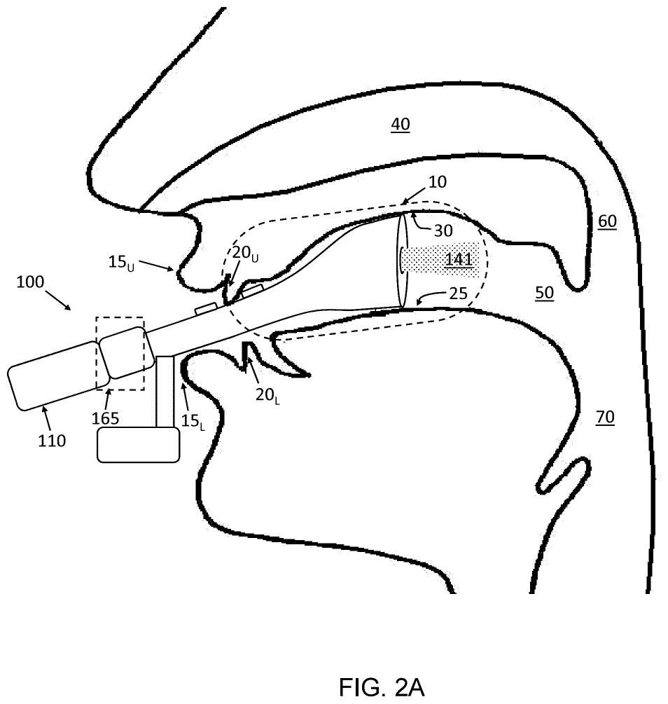

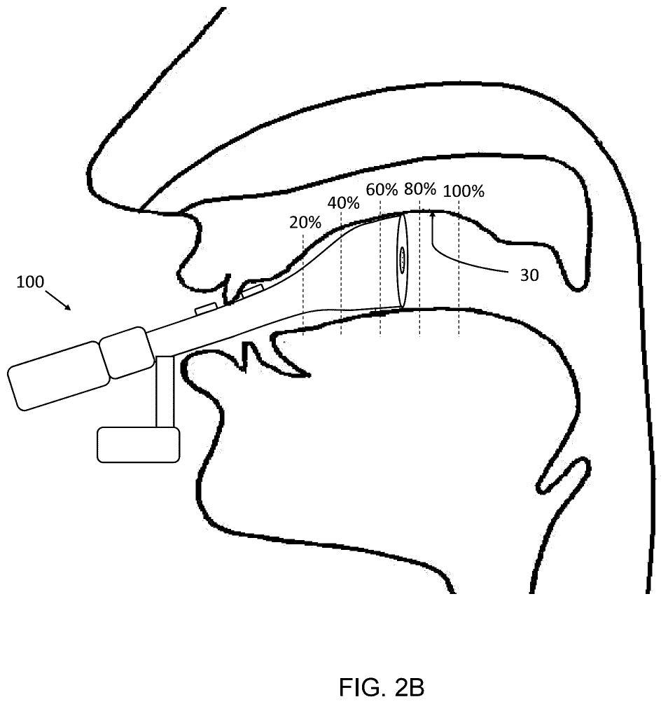

Referring now to FIG. 2A, the in-situ placement of an inhalation device 100 according to embodiments (and according to the example of FIGS. 1A-C) is shown with respect to a user's oral cavity 10 and mouth parts such as upper and lower teeth 200, 20L, upper and lower lips 150, 15L, tongue 25 and hard palate 30. The inhalation device 150 is preferably dimensioned such that the distal portion 175 spans the oral cavity 10 from the tongue 25 to the hard palate 30. Teeth 20 and/or lips 15 can close on the device 100 at teeth-engaging portions or lip-engaging portions that are distally displaced from the proximal portion 165, and thus help to maintain the position of the device 100 as illustrated. The comfort elements 123 illustrated one non-limiting example of a teeth-engaging portion. Mist 141, as shown schematically in FIG. 2A, is produced at the mesh membrane 185. The terms ‘mist’ and ‘aerosol’ are used and understood interchangeably. The generated aerosol is mixed into the inhaled air by the human user and carried into the bronchial tree and the lungs; as mentioned hereinabove, the mist-generating location and mist-exiting location (i.e., the aerosol exit from inhaler 100) are the same location in this exemplary design and are generally to be understood as one and the same as the aerosol generating location. In other words, not only is the aerosol outlet located in the distal portion 175 of the inhalation device 100, in this design the mesh membrane 185 is also located in the distal portion 175. Thus when positioned as illustrated with the top surface of 178 of the distal portion 175 in contact with the hard palate 30, and the bottom surface of 176 of the distal portion 175 in contact with the tongue 20, both the mesh membrane and the aerosol exit are placed in fluid communication with the user's oropharynx 50. A proximal air inlet (not shown in FIG. 2A) can be added for ensuring that proper inhalation can still occur when lips 15 are closed around the device 100.

In embodiments, the distal portion 175 of the inhalation device 150 can comprise a coating for generating a taste and/or odor sensation for the user. The coating can be applied, for example, on the tongue-contacting portion of the distal portion.

In another design example, which is not illustrated, the inhaler 100 of FIGS. 1-2 can be formed to be shorter, so that the container 110 is part of the proximal portion and the teeth-engaging and/or lip-engaging location is on a surface of the container 110.

In embodiments, the distal portion 175 of the inhalation device 150 includes the mist-generating location, i.e., the mesh membrane 185, and the device 150 is formed so that the mist-generating location is beneath the hard palate 30. In some embodiments, the volume of the oral cavity beneath the hard palate can be demarcated according to ‘hard-palate-deepness’ as illustrated schematically in FIG. 2B. For example, the mist-generating location can be in the deeper half of the volume beneath the hard palate 30—or in the deepest 40% in the example of FIG. 2B, or in the deepest 20% (not shown). In other words, the mist-generating location (and the mist-exiting location) can be at least 50% deep into the volume beneath the hard palate 30 or at least 60% deep or at least 70% deep or at least 80% deep. In other embodiments, the mist-generating location might not be quite as deep—for example, the mist-generating location can be at least 20% deep or at least 30% deep or at least 40% deep into the volume beneath the hard palate. Greater ‘deepness’ can be advantageous so as to shorten the path of fluid communication between the mist-exiting location and the oropharynx.

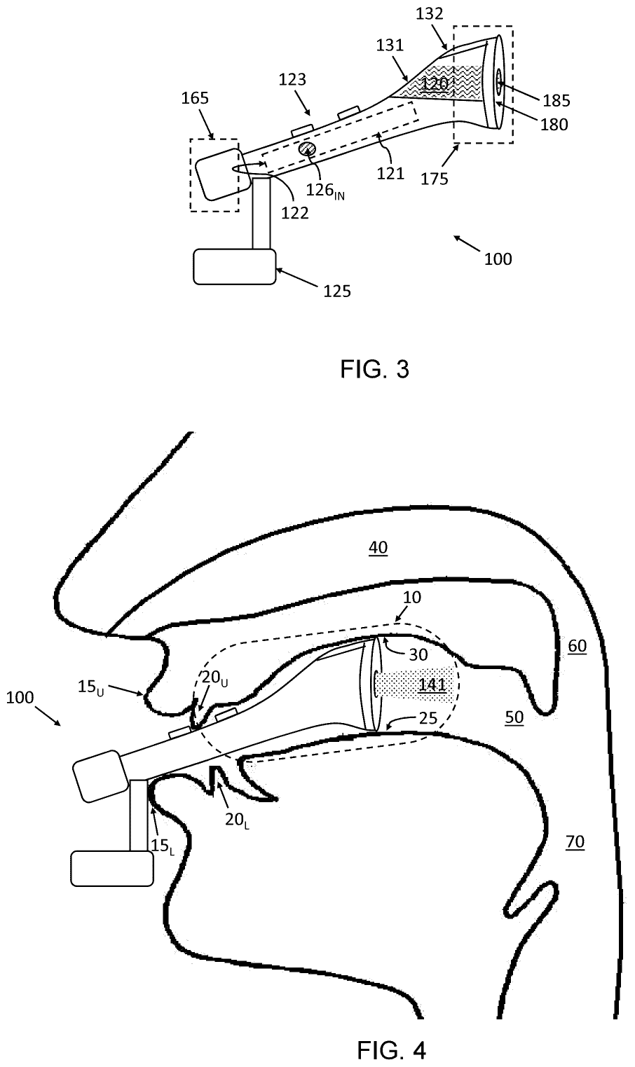

We now refer to FIGS. 3 and 4.

FIG. 3 illustrates a more compact design for an inhalation device 100 according to embodiments of the present invention, and FIG. 4 shows the in-situ placement of the device 100. Like the inhaler 100 of FIGS. 1A-2, the inhaler 100 of FIGS. 3 and 4 has a distal portion 175 (comprising the piezo assembly 180 and the aerosol outlet which happens to be co-located with mesh membrane 185) and a proximal portion 165, power and electronics module 125, and optional comfort elements 123. Liquid 120 for producing therefrom a mist is stored in compartment 131 (which is optionally detachable/attachable). Compartment 131 has a opening for filling and refilling; the compartment 131 has an openable closing element 132. The inhalation device 100 of FIG. 3 includes an airflow channel 121 having a proximal air inlet 122 for ensuring that proper inhalation can still occur when lips 15 are closed around the device 100. As can be seen in FIG. 4, the air inlet 122 is positioned so as to remain outside the lips 15 when the device 100 is positioned for operation in situ. Referring again to FIG. 3, an ‘inhalation sensor’, i.e., flowmeter or airflow sensor 126_{IN} is provided for activating the piezo assembly 180 upon detection of inhalation. In embodiments, a piezo assembly 180 can be activated to produce a mist (in the presence of liquid) manually, e.g., by control circuitry in response to a user pressing a button or moving a switch, and/or automatically by control circuitry (e.g., in power and electronics module 125) monitoring the inhalation sensor 126_{IN} for indication of an inhalation airflow. In some embodiments, the inhalation sensor 126_{IN} is configured to detect an air pressure. In some embodiments, the inhalation sensor 126_{IN} is configured to detect a difference between an air pressure in the inhalation flow-path and an ambient air pressure outside the inhalation device

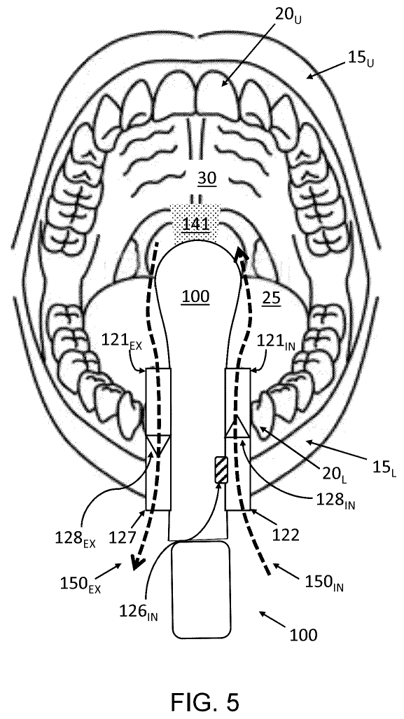

Referring now to FIG. 5, an inhalation device 100 is shown in-situ from a different angle than that of FIGS. 2A-B and 4. Two airflow channels 121, 129 are provided for conveyance of an inhalation airflow (indicated by arrow 150_{IN}) and an exhalation airflow (indicated by arrow 150_{EX}), respectively. As also shown in FIG. 3, the inhalation airflow-channel 121 of FIG. 5 includes an air-inlet 122 positioned beyond user's lips 15 outside of a potentially closed mouth. Similarly, the exhalation airflow-channel 129 includes an exhaust outlet 127 positioned beyond the user's lips 15. In embodiments, each of the airflow channels 121_{IN}, 121_{EX} can be equipped with respective one-way fluid valves 128_{IN}, 128_{EX} which by their presence define the directionality of airflow within each respective airflow-channel. An inhalation sensor 126_{IN}, e.g., a flowmeter or air-pressure sensor, can be provided for monitoring and detecting the presence of an inhalation breath, so that control circuitry can activate or deactivate or otherwise modify the mist-generation of the mesh membrane 180. In embodiments, the mesh membrane can be effective to eject at least 5 times, or at least times, or at least 20 times, or at least 50 times more liquid 120 in the mist 141 during user inhalation than during user exhalation. It will be apparent to those skilled in the art that it does not matter which of the airflow channels 121 is used for inhalation and which is used for exhalation, and the labeling in the figures is merely for convenience.

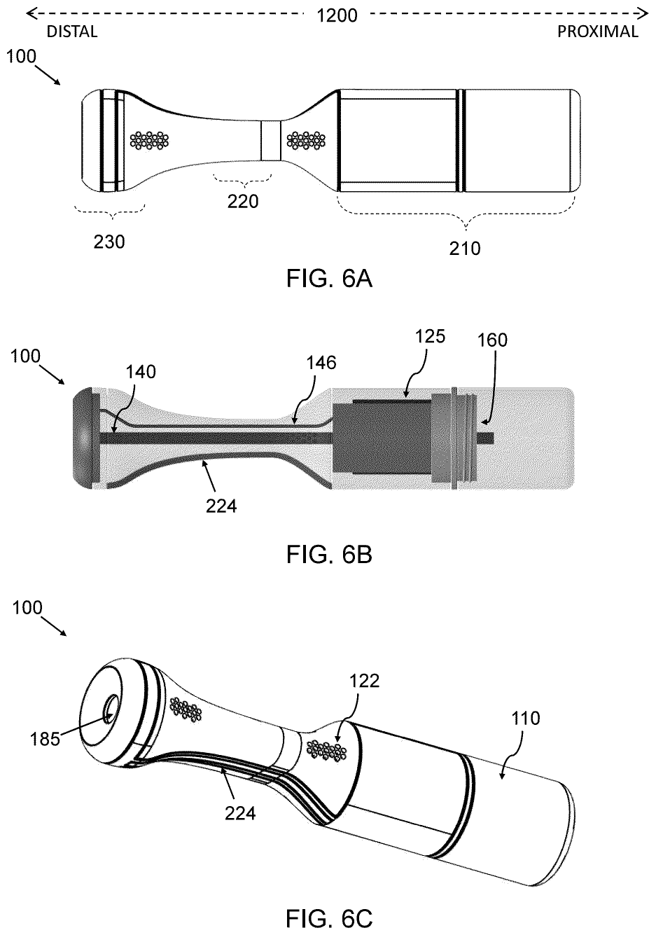

We now refer to FIGS. 6A to 6C, which show various schematic views of an inhalation device 100 according to embodiments, including embodiments already described hereinabove. Respective distal and proximal directions are illustrated throughout by arrows 1200 or 1201, depending on direction—arrow 1200 is shown when distal is to the left, and arrow 1201 is shown when distal is to the right.

As seen in the elevation view of FIG. 6A, the inhalation device 100 includes a distal portion 230, a proximal portion 210 and an intermediate portion 220 that is displaced proximally from the distal portion 230 and distally from proximal portion 210. The proportions of the respective portions 210, 220, 230 are entirely for illustration purposes only, and any of the respective portions 210, 220, 230 can be larger or smaller. In some examples, they can also be contiguous, i.e., without gaps between the various portions. When the inhalation device 100 is in use according to preferred use modes, the intermediate portion 220 is contacted, i.e., transversely engaged, by a user's lips 15 and or teeth 20, and the distal portion 230, which includes the mist-generating mesh 185, is disposed within the user's oral cavity 10.

FIG. 6B, where the outer envelope of the inhalation device 100 is made ‘transparent’, schematically illustrates typical internal components of the inhalation device of FIG. 6A: capillary pathway 140 leading from liquid inlet 160 (where a container or compartment, not shown, would hold a quantity of a liquid) to the mesh 185, and electrical wire(s) 146 leading from control circuitry and power supply 125 to the mesh 185.

As shown in FIG. 6C, the inhalation device 100 can include air-inlet holes 122 which are proximal of the intermediate portion 220 such that the air-inlet holes 122 are outside the mouth. A taste-producing surface section 224 can be provided in the distal portion 230 and/or the intermediate portion 220. The taste-producing surface section 224 is preferably on the ‘bottom’ of the inhalation device 100 during use so as to bring the taste-producing surface section 224 into contact with the user's tongue 25.

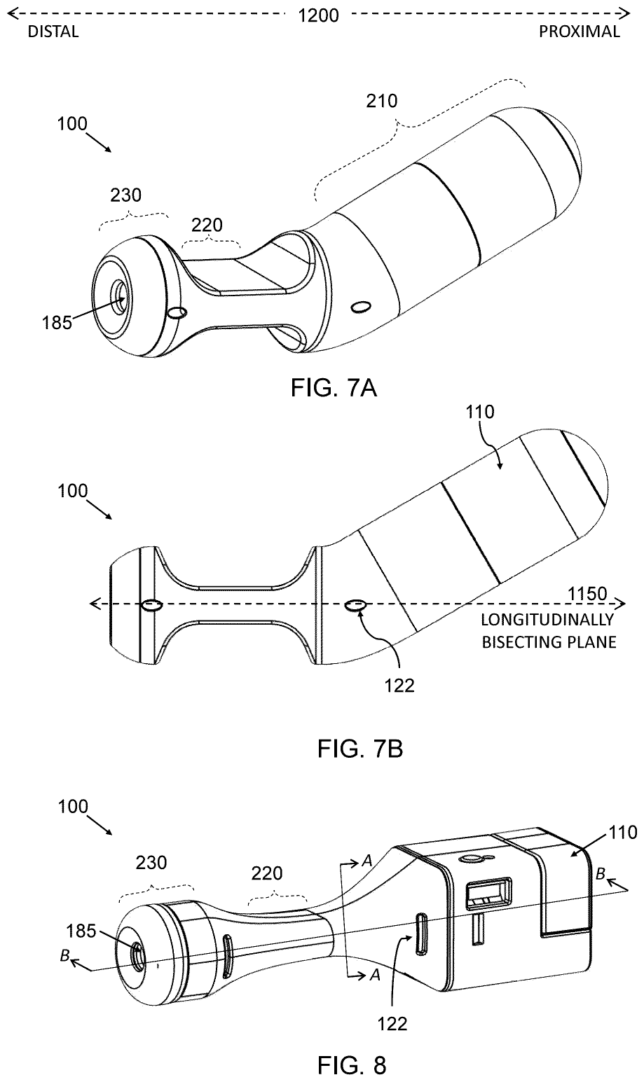

Referring now to FIGS. 7A and 7B, another inhalation device 100 according to embodiments is illustrated. The inhalation device of FIGS. 7A-B can operate effectively without a capillary pathway for transport of liquid from the container 110 to the mesh 185 because of a gravity-aided design. A plane 1150 is shown longitudinally bisecting the intermediate portion 220 (and/or the distal portion 230). When the horizontally-bisecting plane 1150 is held horizontal, e.g., parallel to a floor (not shown), the container 110 is held higher than the plane 1150 and therefore higher than the mesh 185, so that liquid can be made by gravity to flow to the mesh 185. While FIG. 7B shows the entire container 110 as being higher than the plane, in some designs it can be that a portion of the container higher than the plane.

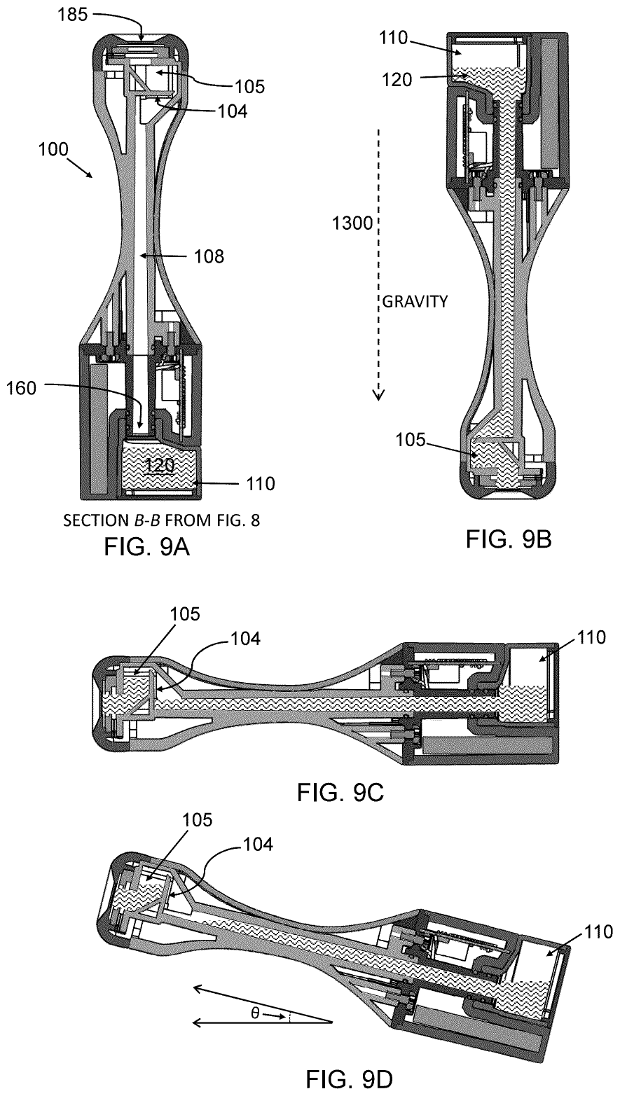

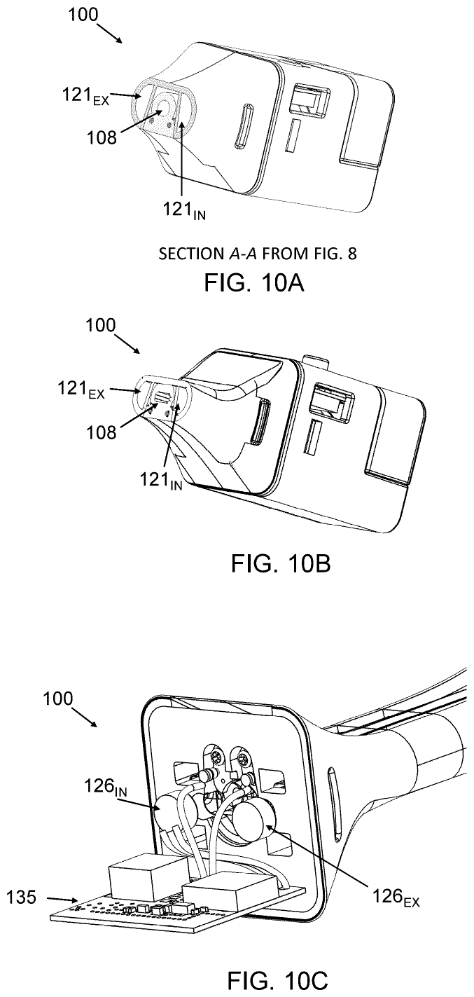

FIG. 8 illustrates another inhalation device 100 according to embodiments, wherein the container 110 does not extend across the entire proximal portion 210 of the inhalation device 100. Features of the inhalation device 100 of FIG. 8 according to embodiments, are illustrated in the cross-sectional views of FIGS. 9A-D, which correspond to section B-B in FIG. 8. In FIG. 9A, a quantity of liquid 120 is disposed in a container 110 which is engaged with liquid-inlet 160. The distal portion 230 includes a distal liquid-retaining fluids compartment 105 in fluid communication with the ultrasonic membrane 185. In the embodiment of FIG. 9A, the liquid-retaining compartment 105 in fluid communication is with the proximal liquid inlet 160 vis a liquid conduit 108, illustrated in FIGS. 9A-D as a connecting tube or pipe. The liquid-retaining compartment 105 is partially bounded on one side by a liquid-retaining wall 104. When the inhalation device 100 is turned upside-down as shown in FIG. 9B, the liquid 120 flows down with gravity (indicated by arrow 1300) to fill the liquid-retaining compartment 105, as well as at least a portion of the liquid conduit 108. While FIG. 9B shows the entire liquid conduit 108 full of liquid, and a portion of the liquid remaining in the container 110, in other examples there can be more or less liquid 120 provided, and/or the relative capacities of the retaining compartment 105, the liquid conduit 108 and/or the container 110 can be larger or smaller than illustrated in FIG. 9B such that the liquid 120 fills the liquid-retaining compartment 105 but only some or none of the liquid conduit 108, such that the container 110 is emptied in such examples.

FIG. 9C illustrates the function of the liquid-retaining wall 104 that partially bounds the liquid-retaining compartment fluids 105. After the reversing of the inhalation device as shown in FIG. 9B, the inhalation can be brought to a horizontal position for use as shown in FIG. 9C, which causes the liquid in the connected reservoirs of the liquid-retaining compartment 105 and the container 110 to tend to ‘seek its own level’. However, the liquid-retaining wall 104 prevents a portion of the liquid 120 delivered to the liquid-retaining compartment 105 (during the reversing of the inhalation device 100) from leaving the liquid-retaining compartment 105 after the inhalation device 100 is turned horizontal, or, as illustrated in FIG. 9D, ‘below horizontal’. The liquid 120 in the liquid-retaining fluids compartment 105 can thus be ‘cut off’ from the remainder of the liquid in the container 110 and conduit 108. The height of the liquid-retaining wall 104 is preferably sufficient to ensure that for a range of angles 0 (horizontal, e.g., as in FIG. 9C) to θ (e.g., as in FIG. 9D), the mesh 185 is kept in contact with liquid 120 retained in the liquid-retaining compartment 105. Setting the value of θ is a design choice which reflects a desired range of angles at which the inhalation device 100 can work effectively. The height of the liquid-retaining wall 104 should be sufficient to retain liquid 120 in the compartment 105 through the range of angles 0 to θ even though the surface level of the liquid 120 in the compartment 105 is higher than in the container 110, as illustrated in the example of FIG. 9D. During normal operation, whenever enough liquid 120 is nebulized out of the inhalation device to cause a portion or substantial portion, e.g., over 1 mm, of the mesh 185 to no longer be in contact with liquid 120, the user can simply upend the inhalation device (as in FIG. 9B) to ‘refill’ the liquid-retaining compartment 105 from the remaining liquid 120 in the container 110 and conduit 108, and then restore the comfortable use position of FIG. 9C or FIG. 9D.

In embodiments, a maximum retainable fluid capacity of the liquid-retaining compartment fluids 105 (i.e., the quantity of the liquid 120 retained by the liquid-retaining wall 104) is at least 0.2 cc and not more than 4 cc. In some embodiments, the maximum retainable fluid capacity of the liquid-retaining compartment 105 is at least 0.5 cc and not more 3 cc. In some embodiments, the maximum retainable fluid capacity of the liquid-retaining compartment 105 is at least 0.5 cc and not more 2.0 cc. A ratio of (i) a combined fluid capacity of the container 110 and the conduit 108 to (ii) the maximum retainable fluid capacity of the liquid-retaining compartment 105, is at least 1 and not more than 4. In some embodiments, this ratio is at least 1.5 and not more than 3. In some embodiments, this ratio is at least 1.75 and not more than 2.5.

Reference is made to FIGS. 10A, 10B and 10C, both of which show cross-sectional views corresponding to section A-A in FIG. 8 such that the liquid conduit 108 and respective airflow channels 121_{IN}, 121_{EX} can be seen. The liquid conduit 108 of FIG. 10A has a circular cross-section. The liquid conduit 108 of FIG. 10B has an oval cross-section, which, inter alia, can be effective to reduce turbulent flow within the liquid conduit 108.

FIG. 10C shows respective inhalation and exhalations sensors 126_{IN}, 126_{EX}. When present each of the sensors 126 is in communication with a respective flow path 121. In some embodiments, only one of the sensors 126 is present.

The inhalation sensor 126_{IN} is provided for monitoring a flow in an inhalation flow-path, e.g., inhalation flow path 121_{IN}. In an example, the inhalation sensor 126_{IN} can be effective to detect an air pressure in the inhalation-flow path 121_{IN}. In another example, the inhalation sensor can be effective to detect a difference between an air pressure in the inhalation flow-path 121_{IN} and an ambient air pressure outside the inhalation device 100. In some embodiments, the control circuitry 135 is configured to initiate and/or cease activation of the mesh membrane 185 in response to a result of the monitoring of the flow in the inhalation-path 121_{IN}.

The exhalation sensor 126_{EX} is for monitoring a flow in an exhalation-flow path, e.g., exhalation flow path 121_{EX}. In an example, the exhalation sensor 126_{EX} is configured to detect a concentration of a chemical compound in the exhalation-flow path 121_{EX}. In some embodiments, the chemical compound is a component of the liquid 120 which is misted by the inhalation device. In some embodiments, the chemical compound is a chemical compound of interest to a user. For example, a user may wish to know the concentration of an intoxicating chemical compound in an exhalation, such as, for example, and not exhaustively, alcohol or tetrahydrocannabinol. In another example, the chemical compound can be an indicator of a disease or of a current health condition of the user. In some embodiments, the control circuitry 135 is configured to cease or delay activation of the mesh membrane 185 in response to a result of the monitoring of the flow in the exhalation flow path 121_{EX}.

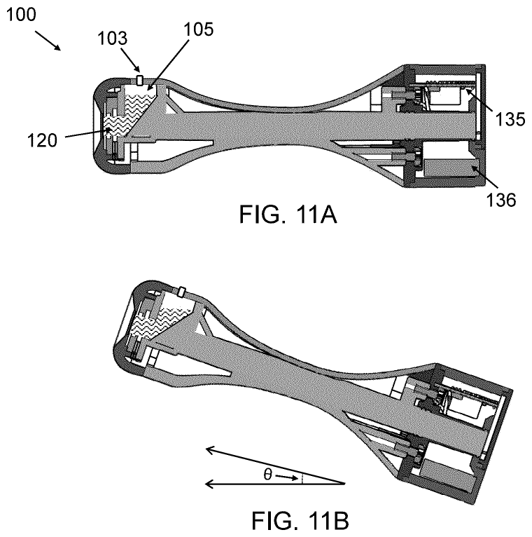

Referring now to FIGS. 11A and 11B: an inhalation device 100 comprises a distal fluids compartment 105 is in fluid communication with the ultrasonic membrane 185. In an embodiment as shown in FIGS. 11A and 11B, the fluids compartment 105 is fillable through filling port 103. In the design of FIGS. 11A and 11B, the inhalation device 100 does not include a proximal source of liquid 120, nor does it include a liquid conduit 108. Instead, the liquid in the distal fluids compartment 105 is in contact with the mesh membrane. The distal fluids compartment 105 is designed such that for a range of angles 0 (horizontal, e.g., as in FIG. 11A) to θ (e.g., as in FIG. 11B), the mesh 185 is kept in contact with liquid 120 retained in the fluids compartment 105. Setting the value of θ is a design choice which reflects a desired range of angles at which the inhalation device 100 can work effectively.

It can be desirable to add a display screen (or, equivalently, any display device) to an inhalation device for visually communicating information to a user. The information to be communicated can include, for example, and not exhaustively: the quantity or percentage of liquid remaining; the quantity or percentage of a compound in the liquid that is remaining; the amount or percentage of liquid (or of the compound in the liquid) that has already been consumed by the delivery of the mist, with or without including prior any fills of the liquid; the identity of the compound; a power meter showing remaining battery life; a concentration of a compound detected in an exhalation airflow; whether a concentration of a substance in the exhalation airflow exceeds a preset limit for intoxication; and a health indicator such as the presence of a virus, bacteria, or any other health indicator that can be detected in an exhalation.

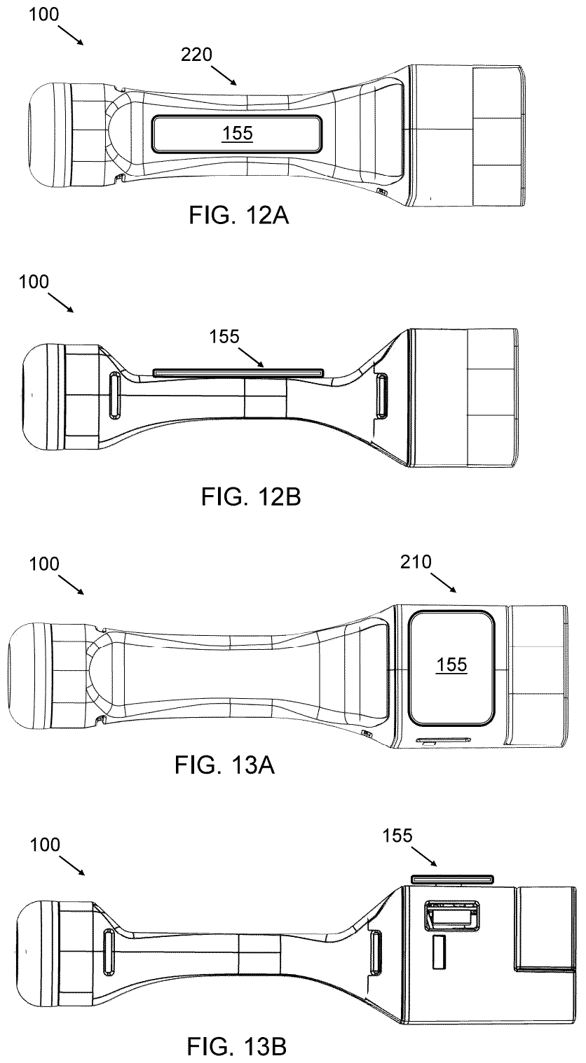

A display screen can be mounted to or installed on any convenient section of any of the inhalation devices 100 disclosed herein. FIGS. 12A and 12B (top and ide views, respectively of an inhalation device 100 according to embodiments) show a display screen 155 mounted to the intermediate portion 220 of the inhalation device 100. FIGS. 13A and 13B (top and ide views, respectively of an inhalation device 100 according to embodiments) show a display screen 155 mounted to the proximal portion 210 of the inhalation device 100.

In any of the embodiments disclosed herein, the liquid 120 can include a medicament. In some embodiments, the quantity of liquid 120 used to generate the mist 141 can be a based on a predetermined dosage. This can be accomplished by the control circuitry in accordance with previous programming or in response to a user input.

In any of the embodiments disclosed herein, a capillary pathway 140 may be used to transport liquid to the mesh membrane.

In any of the embodiments disclosed herein, the inhalation device 150 can be used ‘hands-free’, i.e., when the inhalation device 150 is disposed so that the user's teeth are engaged with a front-teeth-engaging portion distally displaced from the proximal portion 165, and/or the user's lips are engaged with a lip-engaging portion distally displaced from the proximal portion 165, the device 150 can be held in place by the user's lips 15 and or teeth 20 during activation/operation and mist-generation without having to use a hand to keep it in place.

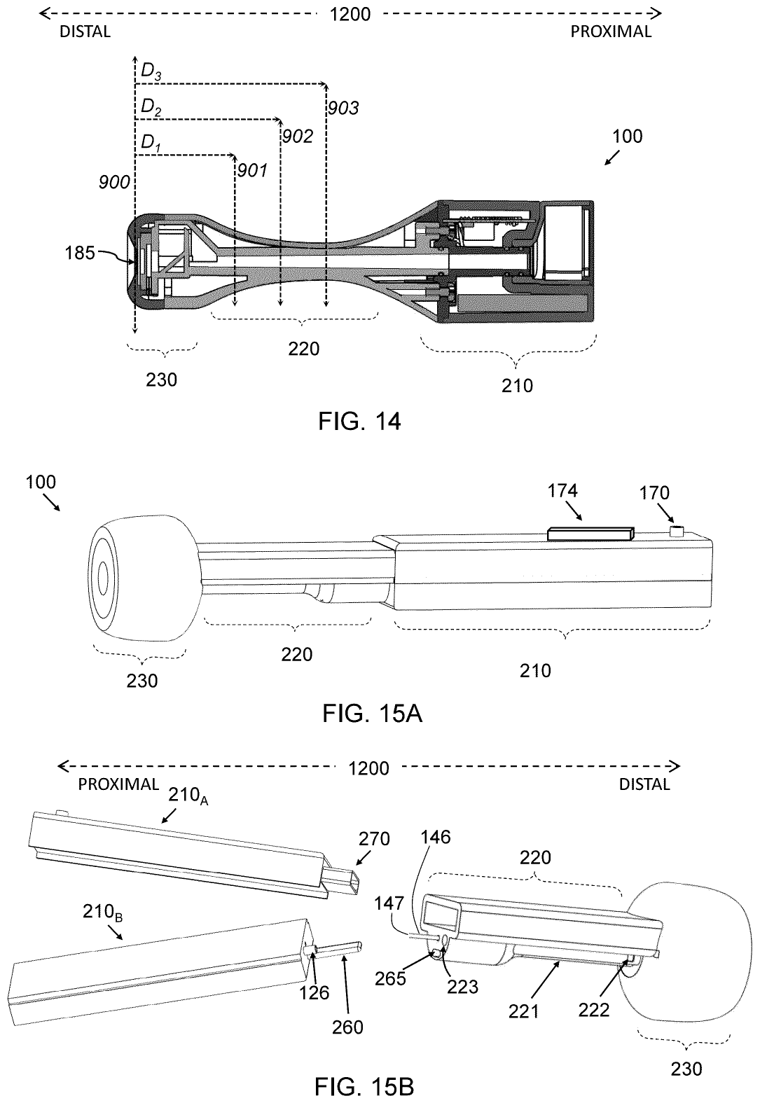

We now refer to FIG. 14. An inhalation device 100, e.g., the inhalation device 100 of FIGS. 9A-9D, is illustrated with annotations marking certain locations and dimensions according to embodiments. A plane indicated by arrow 900 passes through the mesh membrane 185 and is parallel thereto. A cross section of the distal portion 230 and through the mesh membrane 185 can be round, oval, or any other shape. A minimum dimension of the cross-section, i.e., measured by an arc or line segment that passes through a center-point of the cross-section is a minimum cross-sectional dimension of the distal portion 230 passing through and parallel to the mesh membrane 185. The mesh membrane, in embodiments, is encompassed, at least circumferentially, by a distal casing 190. A neck portion 220 of the inhalation device 100, analogous to the intermediate portion 220 shown, e.g., in FIGS. 6A and 7A, is located proximal to the distal portion 230. In some designs, the neck portion 220 is contiguous with the distal portion 230 and in some designs there can be additional inhaler length in between that for purposes of this disclosure has the distinguishing features of neither the distal portion nor the neck portion, and can be arbitrarily assigned to either one.

The neck portion 220 includes at least one narrow section. A narrow section can comprise a single point or a longer length of the neck portion 220. A narrow section is characterized by having a cross-section with a minimum dimension that is smaller, by a given margin, than the minimum cross-sectional dimension of the distal portion 230 passing through and parallel to the mesh membrane 185. All uses of the term ‘cross-sectional dimension’ herein refer to external cross-sectional dimensions unless explicitly stated otherwise. For example, a minimum cross-sectional dimension in a narrow section can be at least 10% smaller than the minimum cross-sectional dimension of the distal portion 230 passing through and parallel to the mesh membrane 185. Any given point in a narrow section need not be the narrowest point of the inhalation device/system. The can be a narrowest point, which is the point with the smallest minimum cross-sectional dimension in a narrow section, i.e., in the inhalation device/system. In other examples, the minimum cross-sectional dimension in a narrow section can be at least 20% smaller, or at least 30% smaller, or at least 40% smaller, or at least 50% smaller, or even smaller. The minimum cross-sectional dimension of the narrow section and the minimum cross-sectional dimension of the distal portion 230 define vectors that are coplanar, or within ±15° of being coplanar, or within ±30° of being coplanar, or within ±45° of being coplanar.

Three examples of narrow sections are shown in FIG. 14 where planes indicated by the arrows 901, 902 and 903 pass through the neck portion 220. Additionally or alternatively, the entire neck portion 220 can be considered as comprising one narrow section from one end of the neck portion 220 to the other, because in FIG. 14 the entire neck portion 220 clearly can be seen to be characterized by a minimum cross-sectional dimension that is at least 10% smaller than the minimum cross-sectional dimension at the plane 900 of the distal portion 230 passing through and parallel to the mesh membrane 185. In this case, the first, i.e., distalmost point of the ‘narrow section’ meeting the 10%-smaller limitation can be is of interest, although in some embodiments all points of the narrow sections of the neck portion 220 can be of interest. For example the first (distalmost) annotated point in the neck portion 220 is defined by a cross-section at plane 901. As shown in FIG. 14, the plane 901 is proximally displaced from the mesh membrane 185, i.e., from the plane 900 passing through and parallel to the mesh membrane 185, by a distance indicated by the arrow D₁. In embodiments, this distance D₁ is at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. Similarly, if the narrow section at plane 902 were to be the first (distalmost) point meeting the 10%-smaller limitation, then the corresponding distance from the mesh membrane 185 (and plane 900) D₂ would be, in such a design, at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. Similarly, if the narrow section at plane 903 were to be the first (distalmost) point meeting the 10%-smaller limitation, then the corresponding distance from the mesh membrane 185 (and plane 900) D₃ would be, in such a design, at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. In other words, the first (distalmost) point in the narrow section, i.e., the distal end of a narrow section, which meets the 10%-smaller limitation is at a distance from the mesh membrane 185 (and plane 900) that falls in one of the ranges above, i.e., at least 0.5 cm and not more than 5.5 cm, or at least 0.5 cm and not more than 5 cm, or at least 0.5 cm and not more than 4.5 cm, or at least 0.5 cm and not more than 4 cm, or at least 1 cm and not more than 6 cm, or at least 1 cm and not more than 5.5 cm, or at least 1 cm and not more than 5 cm, or at least 1 cm and not more than 4.5 cm, or at least 1 cm and not more than 4 cm. In the foregoing, the 10%-smaller limitation is used as a non-limiting example, and in some embodiments the minimum cross-sectional dimension in a narrow section can be at least 10% smaller than the minimum cross-sectional dimension of the distal portion 230 passing through and parallel to the mesh membrane 185.

In embodiments, the inhalation device 100 is designed so that a center of gravity of the inhalation device 100 is proximal to the first (distalmost) point in the narrow section i.e., the distal end of a narrow section, which meets the 10%-smaller limitation, when the inhalation device 100 contains no liquid.

Referring now to FIGS. 15A and 15B, an inhalation device according to embodiments is schematically illustrated, respectively assembled and unassembled.

As shown in FIG. 15B, this design can be supplied in a modular configuration, where two proximal modules 210A, 210B are detachably attachable to/from an inhalation-device section (shown in FIG. 17B as 450) comprising both the distal portion 230 of the inhalation device 100 and the neck portion 220 of the inhalation device 100. A first proximal portion 210A includes an interior liquid-holding volume (not shown) and can be used as a replaceable container for the inhalation device 100.

The first proximal portion 210A includes, according to some embodiments at least one of a liquid inlet 170 and a pressurable surface 174, e.g., a flexible surface that can be manually depressed so as to cause a liquid to flow out of the first proximal portion 210A and into the neck portion 220 of the inhalation-device section 450. In some embodiments, the outlet 270 of the first proximal portion 210A includes a pressure-activated one-way valve such as, in a non-limiting example, a duckbill valve. In some embodiments, at least an upper surface of first proximal portion 210A is flexible and a separate pressurable surface 174 is unnecessary, and in some embodiments, the entire first proximal portion 210A is flexible and a separate pressurable surface 174 is unnecessary.

An electronic and/or electrical connection 146 inserts into a corresponding hole (not shown) in the second proximal module 210B for powering the piezo assembly 180 from a power source 125 located in the second proximal module 210B. In embodiments, an inhalation sensor 126 for indication of an inhalation airflow is provided on the second proximal module 210B. In the assembled state, the inhalation sensor 126 is in fluid communication with the end 223 of an airflow channel 221 having a distal air inlet 222 located proximal to the distal portion 230. Thus, when a user holds the inhalation device in his mouth, e.g., as illustrated in FIGS. 2A-2B, mutatis mutandis, and the user's lips 15 close around the neck portion 220 of the inhalation device 100, an inhalation will draw air through the distal air inlet 222 and through the air channel 221, causing the inhalation sensor 126 to register a pressure drop and initialize the activation of the piezo assembly 180 and generate a mist. Similarly, the sensor 126 can register the cessation of an inhalation and cease the operation of the piezo assembly and the generation of the mist.

In embodiments, one or more stabilization attachments 260 may be provided for making and stabilizing the connection of the second proximal module 210B and the inhalation-device section 450. In embodiments the attachment is reversible, so as to enable a attachable/detachable link. For example, the stabilization attachments 260 may take the form of a mechanical pin (illustrated example) or a snap or a magnetic attachment as known in the art. The one or more stabilization pins 260 are arranged for being inserted into corresponding hole(s) 265. Additionally or alternatively, other features can be added for making and stabilizing the connection of the second proximal module 210B and the inhalation-device section 450, as well as the connection of the first proximal portion 210A and the inhalation-device section 450.

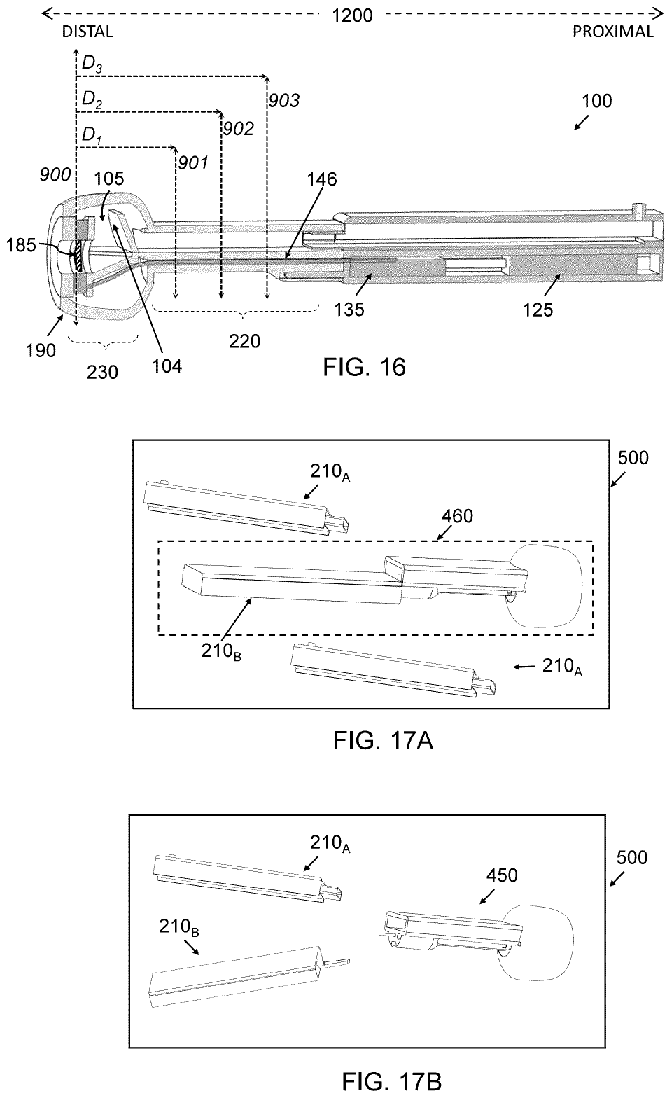

FIG. 16 shows a schematic cross-section of the inhalation device 100 of FIGS. 15A-15B. As can be seen, the inhalation device 100 of FIGS. 15A-15B incorporates many of the features disclosed hereinabove for the various designs of inhalation devices 100, including, and not exhaustively: the liquid-retaining compartment 105 partially bounded on one side by a liquid-retaining wall 104; the definition of narrow sections as discussed with reference to FIG. 14; the distal casing 190 encompassing, at least circumferentially, the mesh membrane 185; and the internal power source 125 and electronic circuitry 135 connected by electric contact 146.

Referring to FIGS. 17A and 17B, the modular components of the inhalation device 100 of FIGS. 15A, 15B and 16 can be provided in a kit, packaged in a container 500. A first example of a kit is shown in FIG. 17A, comprising an inhalation-sensor section 460 that includes the distal portion 230, the neck portion 220, and the second proximal module 210B. The kit also includes a first proximal portion 210A. In some embodiments, the kit of FIG. 17A can include, as shown, at least one additional first proximal portion 210A. A second example of a kit is shown in FIG. 17B, comprising an inhalation-sensor section 450 that includes the distal portion 230 and the neck portion 220, and, unattached, the first and proximal modules 210A, 210B.

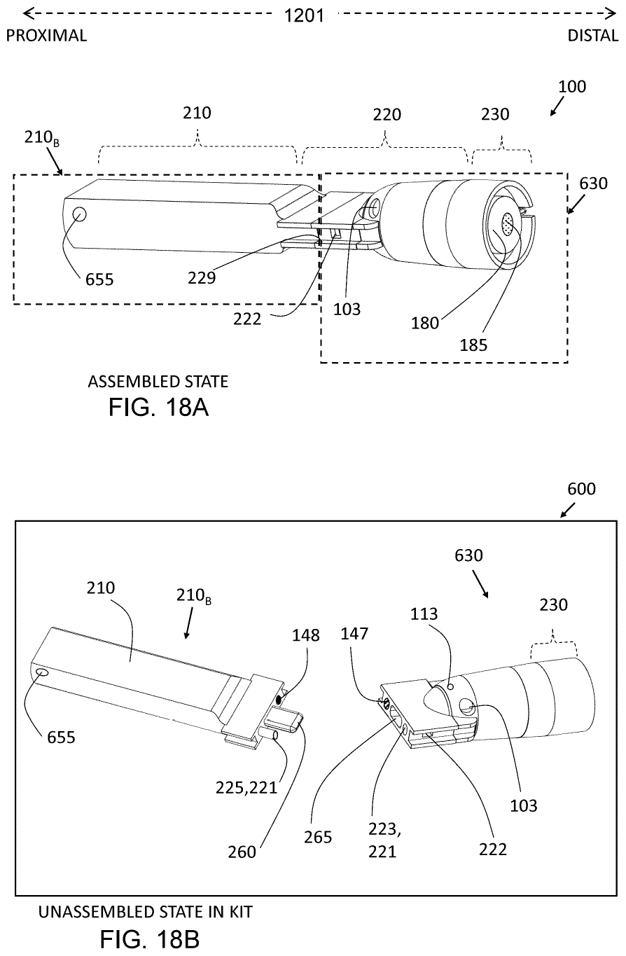

Referring to FIGS. 18A and 18B, illustrated is an embodiment of an inhalation system 100 for delivery of an aerosol to the oropharynx of a human user. The system 100 comprises an aerosol delivery module 630 comprising a device distal portion 230 of the aerosol delivery module 630. The distal portion 230 comprises (i) an aerosol outlet 186 defining a mist-exiting location and (ii) a piezo assembly 180 including an ultrasonically vibrable mesh membrane, which upon electrical activation produces a mist comprising droplets of the liquid, the mesh membrane 185 defining a mist-generating location. The aerosol delivery module 630 further comprising a distal fluids compartment 105 in fluid communication with the mesh membrane 185; the compartment 105 comprising a compartment-proximal-wall 104, and a device mid-section neck portion 220 including a narrow section, the narrow section being characterized by having a location of a narrow width cross-sectional dimension WN that is at least 10% smaller than a minimum width WD dimension of a more distal cross-section at a membrane plane 900 passing through the mesh membrane, the membrane plane 900 being perpendicular to the geometrical axis 910 perpendicular to and passing through the center of the mesh membrane 185.

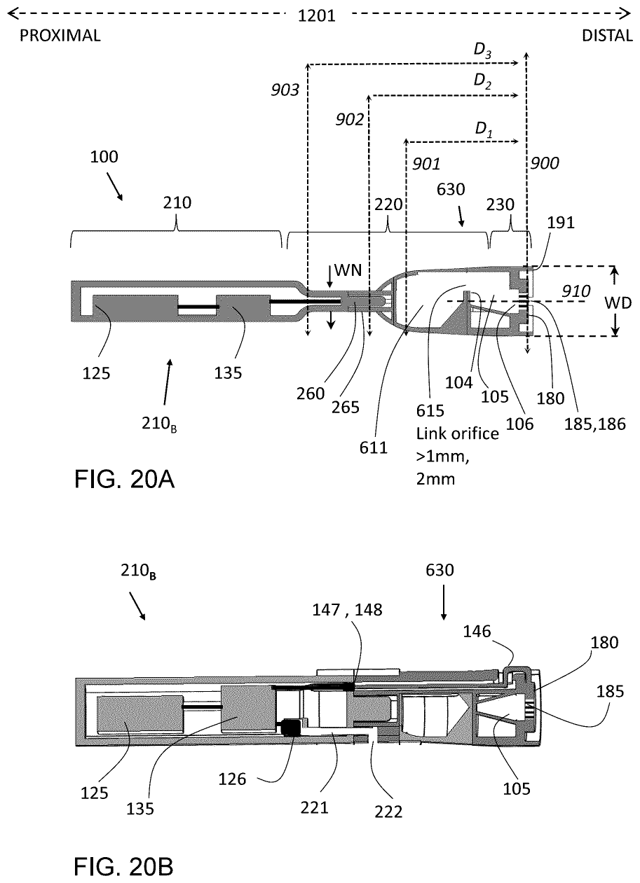

The narrowness condition may be satisfied at more than one location along the aerosol delivery module 630. For example, as illustrated in FIG. 20A, proximally to the distal portion 230 the thickness or width of the system 100 gets gradually narrower along the aerosol delivery module 630, as one progress through cross sections 901 to 902. It is already true that the width at cross section 901 is already less than 90% of the width WD dimension of a cross-section at a membrane plane 900 passing through the mesh membrane. But the width at the cross section 902 is even narrower by a significant amount, as much as 50% or more. There is user comfort and stability advantage to have a significantly narrow portion 220, since the system 100 is intended to be held in use with the distal section 230 being within the mouth and the narrow portion 220 may be engaged with the teeth and/or lips.

In embodiments, the narrow section is characterized by having a location of a narrow width cross-sectional dimension WN that is at least 20% smaller, or at least 30% smaller, or at least 50% smaller, or at least 70% smaller, than a minimum width WD dimension of a more distal cross-section at a plane 900 passing through and tangential to the center of the mesh membrane. In some embodiments, the narrow width cross-sectional dimension WN is of size between 8 mm and 10 mm, or between 6 mm and 8 mm, or between 4 mm and 6 mm, or less than 4 mm.

As illustrated in FIG. 18A the inhalation system 100 comprises a proximal device portion 210, which is proximal to the neck portion 220.

In the embodiment of FIG. 18A, at least part of the neck portion 220 is within the aerosol delivery module 630. The proximal device portion 210 is a part of a control module 210B which may also comprise narrow parts which may or may not be included in the neck portion 220.

FIG. 18A illustrates an embodiment wherein the fluids compartment 105 is in fluid communication with a filling port 103. In some embodiments, the filling port 103 comprises a check-valve, or any other type of one-way valve. In other embodiments the fluids compartment may comprise an openable and re-closeable portion of its shell wall so as to enable insertion of liquids. In some embodiments the openable portion of the shall-wall may be detachable. In some other embodiments the openable portion of the shall-wall may be connected to the fluids compartment, for example on a hinge or by a string or strap.

FIG. 18B illustrates an embodiment wherein the inhalation system has a control module 210B which is attachable and detachable from the aerosol delivery module 630. In the connected configuration state, a full system device 100 is formed. Thus, in an embodiment, system 100 is an assembly combining two main parts, the aerosol delivery module 630 and the control module 210B. The aerosol delivery module 630 comprises the device distal portion 230 of the system distal section which includes (i) an aerosol outlet 186 defining a mist-exiting location and (ii) a piezo assembly 180 including an ultrasonically vibrable mesh membrane. The aerosol delivery module 630 also includes a distal fluids compartment 105 in fluid communication with the mesh membrane 185. Correspondingly, in embodiments, a proximal control module 210B includes a power source 125, the control module 210B further comprising (i) a pressure sensor 126, (ii) an attachment element 260, and (iii) proximal electrical contacts 148.

As illustrated in FIG. 18B, in some preferred embodiments, in addition to the filling port 103 into the liquids chamber, there is a secondary port 113 of smaller cross section than the filling port 103. Port 113 is serving as an air outlet simultaneously with liquids filling via the filling port 103. In preferred embodiments, the cross section area of the filling port 103 is smaller than 1 cm² and bigger than 0.1 cm², or smaller than 0.5 cm². In some preferred embodiments, the cross-section area of the secondary filling port 113 is smaller than 0.3 cm² and bigger than 0.001 cm², or smaller than 0.1 cm².

When in use, the aerosol delivery module 630 resides at least in part within the mouth of the user. In some embodiments, a narrow section at a proximal end of the aerosol delivery module 630 can be outside the mouth, or can include the location where the user's lips and/or teeth can close upon the inhalation system 100. Moreover, the mesh membrane 185 and the fluids compartment 105 are in contact with the liquids which are used. In embodiments, the control module 210B remains outside the mouth and does not come in contact with the liquids put into the distal fluids compartment 105. Therefore, from a cleanliness and maintenance perspective, it may be desirable to replace the aerosol delivery module 630 more often than control module 210B. In terms of cost, the control module 210B may comprise elements more costly than the aerosol delivery module 630 such that a disposable aerosol delivery module 630 can be replaced at a lower cost while maintaining the same control module 210B for multiple uses.

The attachment mechanism in embodiments may be by a proximal mechanical or magnetic attachment element 260 of the control module 210B that is linked to a distal mechanical or magnetic attachment element 265 of the aerosol delivery module 630.

As illustrated, e.g., in FIGS. 18B, 20A, and 20B, in the assembled configuration state, distal electrical contacts 147 are electrically connected to the piezo assembly 180, and where proximal electrical contacts 148 are connected to the power source 125, and wherein in the assembled state of the system the proximal electrical contacts 148 are engaged with the distal electrical contacts 147, establishing an electrical communication between the piezo assembly 180 and the power source 125.

In embodiments, assembling the system 100 by the user comprises the steps of: connecting the control module 210B to the aerosol delivery module 630, such that in the assembled state
(i) there is fluid communication between the distal port 222 to the pressure sensor 126 via a lumen 221;
(ii) a proximal element 260 of the control module 210B is linked to a distal mechanical or magnetic attachment element 265 of the aerosol delivery module 630 by a mechanical or a magnetic attachment force;
(iii) the proximal electrical contacts 148 are engaged with the distal electrical contacts 147, establishing an electrical communication between the piezo assembly 180 and the power source 125.

In any of the embodiments disclosed herein, the inhalation device/inhalation system can be provided in an assembled state, as illustrated, e.g., in FIGS. 1A, 1C, 2A, 2B, 3-5, 6A-C, 7A-B, 8, 9A-D, 11A-B, 12A-B. 13A-B, 14, 15A, 16, 18A, 19, 20A-B, 21 A-B, 22A, 24A, 25A, 28B. Additionally or alternatively, any disclosed inhalation device/inhalation system can be provided in an unassembled state as separate parts or in a kit optionally including multiple components and/or modules, and optionally including one or more containers, as illustrated, e.g., in FIGS. 1B, 15B, 17A-B, 18B, 22B, 23A-D, 24B, 25B-C, and 28A.

In some embodiments, a versatile kit is formed by having available more than one construction of the aerosol delivery module 630 which are connectable and operable with the same proximal device portion 210B that includes a power source 125. For example, this can be useful when some liquid substances are used at small volume, such as less than 0.5 ml (e.g., more expensive drugs), while other applications use liquid substances at higher volumes such as more than 1 ml. These different use scenarios are better served by dedicated aerosol delivery module 630 comprising a liquids compartment 105 of different size or structural form.

As illustrated in FIG. 18A, an air channel 229 facilitates the flow of ambient air in proximity of the port 222. In particular, during use, when the user lips are wrapped around the device mid-portion neck, the air channel 229, along at least part of the side of the device mid-portion neck, creates an unblocked air fluid communication path for inhalation even when the mouth and lips is closed around the system device. This feature prevents the need for the human user to be conscious of actively letting air pass by the device sides when the lips are more naturally closed.

In embodiments, the system further comprises an electronic external indicator module 655 in electronic communication with the electronic control circuitry 135. For example, in some embodiments the indicator module 655 comprises an LED light. In some other embodiments, the indicator module 655 comprises a display screen 155. The indicator module 655 may serve to indicate various operational states of the system. For example, the indicator module is configured to indicated at least one or more of the states of (i) power on, (ii) Bluetooth connection to an external device, (iii) activation of the piezo assembly 180, (iv) battery charging level, (v) battery being charged on, or more. For example, during system activation by inhalation sensing, the electronic control circuitry 135 is configured to change the indicator module 655 display state when the piezo assembly 180 is activated.

As an intra-oral device, the compactness of the aerosol delivery module 630 is important. In some embodiments the minimum width WD dimension of a more distal cross-section, at a membrane plane 900 passing through the mesh membrane, is less than 20 mm, or less than 15 mm, or less than 12 mm, or less than 10 mm.

FIG. 18B also illustrates a preferred embodiment wherein in addition to the filling port 103 into the fluids chamber 105, also a secondary port 113 of smaller cross section than the filling port 103. The port 113 is serving as an air outlet, for release of air from the fluids chamber, simultaneously with liquids filling via the filling port 103. In preferred embodiments, the cross section area of the filling port 103 is smaller than 1 cm² and bigger than 0.1 cm², or smaller than 0.5 cm². In some preferred embodiments, the cross-section area of the secondary filling port 113 is smaller than 0.3 cm² and bigger than 0.001 cm², or smaller than 0.1 cm².

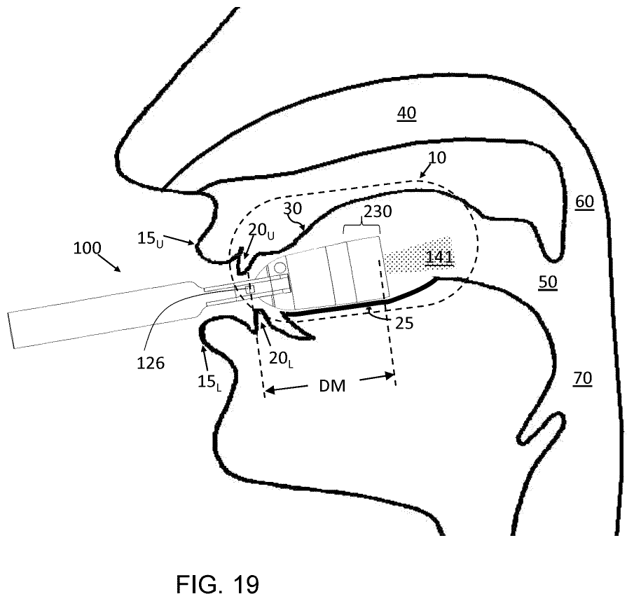

As illustrated in FIG. 19, the inhalation system 100 is shaped such that when the user's lips and/or teeth are transversely engaged with the narrow section, the mist-generating location 185 resides within the user's oral cavity and the mist-exiting location is in direct fluid communication with the user's oropharynx.

In embodiments, as illustrated in FIG. 19, the aerosol delivery module 630 is shaped such that when the user's lips and/or teeth are transversely engaged with a a location of a narrow section, the depth DM of mist-generating location 185 beyond the teeth into the oral cavity is at least 1 cm deep, or at least 2 cm deep, or at least 3 cm deep.

The location of the port 222 is preferably deeper into the oral cavity than the external edge 15L or 15U of the lips. For example, in FIG. 19 the port 222 is shown situated roughly between the teeth.

As illustrated in FIG. 20A, in an embodiment, the inhalation system 100 has an elongated shape such the geometrical axis 910, perpendicular to and passing through the center of the mesh membrane 185, defines a longitudinal axis of the system in the sense that the dimensional extension of the system along the longitudinal axis 910 is larger than the dimensional extension of the system along any axis perpendicular to the axis 910.

In some embodiments, the elongated shape is such that the geometrical axis 910, perpendicular to and passing through the center of the mesh membrane 185, defines a longitudinal axis of the system in the sense that the dimensional extension of the system along the longitudinal a longitudinal-axis oriented similar to the axis 910 is larger than the dimensional extension of the system along any axis perpendicular to the axis said longitudinal axis, wherein said longitudinal-axis is passing through the center of the mesh membrane 185 and is at an angle deviating less than 30 degrees from the axis 910. For example, as illustrated in FIG. 1A, a longitudinal axis 911 passes through the center of the mesh membrane 185 and is at an angle deviating less than 30 degrees from the axis 910.

FIG. 20A shows a schematic cross section of the system 100 in a vertical orientation, illustrating that it includes a power source 125 for electrically powering the piezo assembly 180, the proximal portion is proximal to the distal portion 230, such that more than 50% of the mass of the power source is proximal to the mesh membrane 185.

As illustrated in FIG. 20B, the inhalation system 100 additionally comprises an inhalation sensor 126. In embodiments, the inhalation sensor 126 is in fluid communication with a distal port 222. The inhalation sensor 126 is effective to detect an air pressure difference between an air pressure at the distal port 222 and an ambient air pressure more proximal than the distal port 222.

The inhalation system 100 additionally comprises control circuitry 135. In embodiments, the control circuitry 135 configured to initiate and/or cease activation of the piezo assembly 180 in response to detection of an air pressure difference greater than a threshold limit, and/or cease activation of the piezo assembly 180 in response to detection of an air pressure difference less than a pressure threshold limit. In embodiments the threshold limit is a pressure between 2 cm H2O and 20 cm H2O. In other embodiments the threshold limit is a pressure between 2 cm H2O and 15 cm H2O. In some other embodiments, the threshold limit is a pressure between 5 cm H2O and 15 cm H2O.

As illustrated in FIG. 20A, in an embodiment, the inhalation system comprises a distal fluids compartment 105 bounded by a compartment-proximal-wall 104 which is proximal to the mesh membrane 185, such that the compartment-proximal-wall 104 is extended above and below a height of the line 900 perpendicular to the middle of the of the mesh membrane 185.

In some embodiments, the fluids compartment 105 has a volume of more than 0.1 ml and less than 5 ml, or less than 4 ml, or less than 3 ml, or less than 2 ml, or less than 1 ml.

In embodiments, a center of gravity of the inhalation device is being displaced proximally more than 3 cm from a mesh membrane 185 when the inhalation device is in a liquid-empty state.

As illustrated in FIG. 20B, in some embodiments, the distal port 222 is located distally from the inhalation sensor 126. In embodiments the inhalation sensor 126 is a pressure sensor. In embodiments the sensor 126 is located in the proximal section 210. When the inhalation system 100 is in the assembled configuration state, there is fluid communication between the distal port 222 to the inhalation sensor 126 via a lumen 221, and wherein in the unassembled state a distal portion 223 of the lumen 221 is within the mid-section of the device, and a portion 225 of the lumen 221 is within the device proximal section 210.

The present invention advantages comprise increased use versatility and better economic cost advantages by enabling different level of disposability of various parts. Previously we noted the advantage of the potential cheap disposability of the whole aerosol delivery module 630 with respect to the control module 210B. In embodiments, further versability and disposability is implemented with respect to the fluids chamber 105 component of the system 100 in general and the aerosol delivery module 630 in particular.

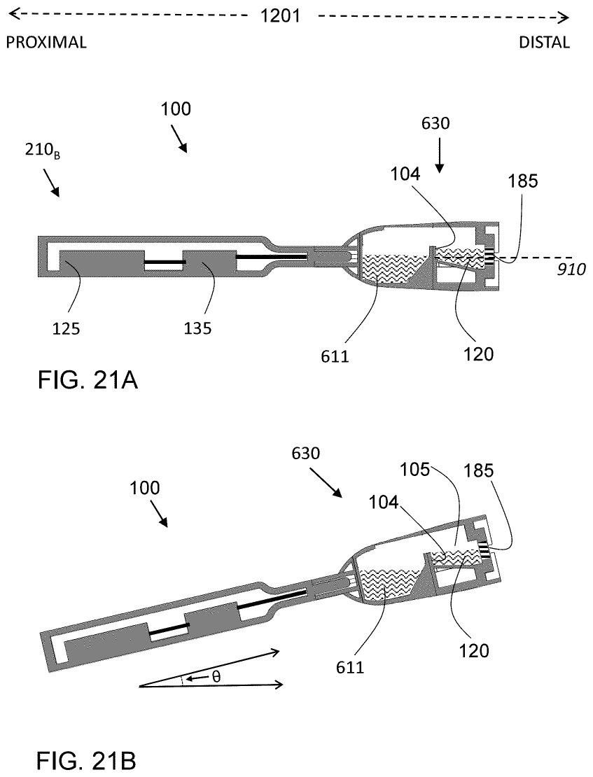

FIGS. 21A and 21B illustrate some aspects of embodiments with a liquid residing within the fluids compartment 105. When compartment 105 is filled with an amount of liquid 120 less than 0.5 ml and the aerosol delivery module 630 is oriented horizontally, such that the axis 910 is perpendicular to the force of gravity, the average surface of the liquid 120 is at a level higher than the middle of the mesh 185.

Moreover, in embodiments structured for use with a small amount of liquid, when compartment 105 is filled with an amount of liquid 120 less than 0.2 ml and the system 100 is oriented horizontally, such that the axis 910 is perpendicular to the force of gravity, the average surface of the liquid 120 is at a level higher than the middle of the mesh 185.

In some embodiments, the system can be functional in a wide range of holding angles with respect to gravity. For example, as illustrated in FIG. 21B, the chamber 105 proximal wall 104 has sufficient extent such that in a second orientation, such that the axis 910 is more than 30 degrees below horizontal, the liquid 120 is in contact with the mesh membrane 185, and the average surface of the liquid 120 at a level higher than 2 mm below the center of the mesh 185.

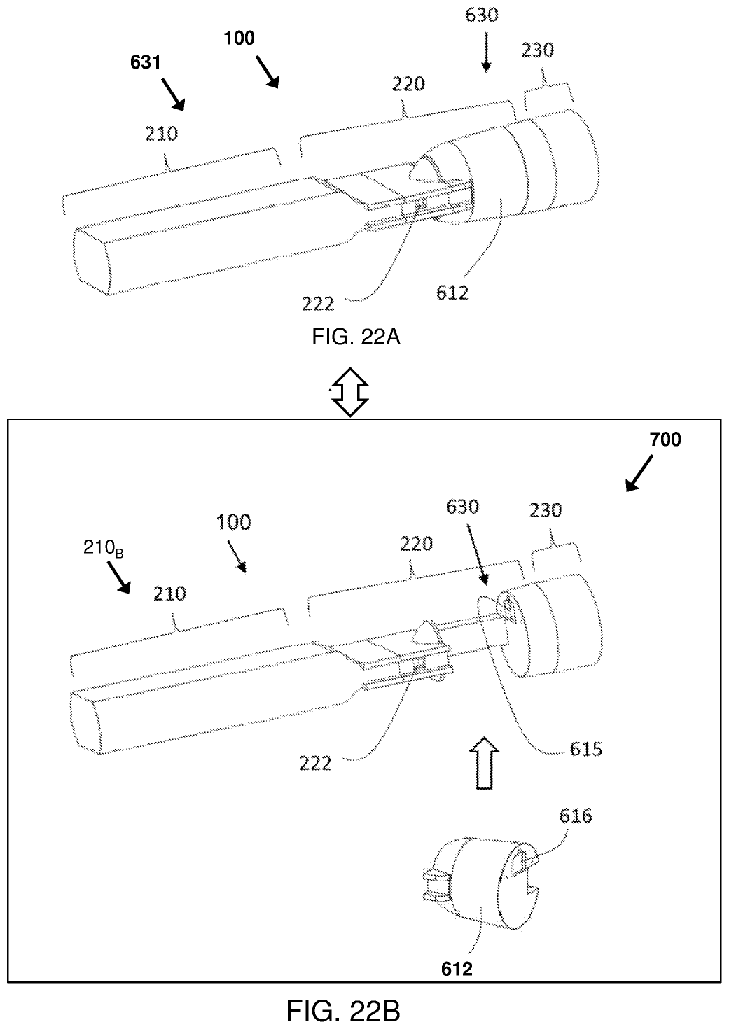

FIGS. 22A and 22B illustrate embodiments further comprising a secondary fluids compartment in fluid communication with the distal fluids compartment 105. The secondary fluids compartment is an independent cartridge device 612 detachably attachable to the distal fluids compartment 105. The cartridge 611 may be prefilled with a liquid 120. In embodiments, the volume of cartridge 611 is larger than the volume of the distal fluids compartment 105.

FIG. 22B illustrates an embodiment wherein the cartridge 611 comprises an outlet port 616, such that in the assembled state the interior of the cartridge 611 is in fluid communication with the distal fluids compartment 105 via outlet port 616.

In the embodiment illustrated in FIG. 22B, the cartridge 611 comprises a hard shell, preferably fitting smoothly with the contours of the surface of the inserted location within the aerosol delivery module 630. To enable better transfer of liquids from the secondary cartridge 611 to the proximal volume of the fluids compartment 105, the aperture of outlet port 616 across the center of the aperture is preferably larger than 2 mm and less than 5 mm.

The cartridge 611 is side-mounted in some embodiments of the assembly process into the aerosol delivery module 630, as illustrated for example in FIG. 22B. Side-mounting is in the sense of inserting motion directed primarily in a direction perpendicular to the longitudinal axis 910. Such side mounting embodiment is not meant to be limiting. A parallel sliding mounting can also be realized in some other embodiments. For example, FIG. 17B illustrates an embodiment of parallel axis mounting of a proximal module 210A. A similar method of mounting can be realized also for the mounting of the cartridge 611 into the aerosol delivery module 630.

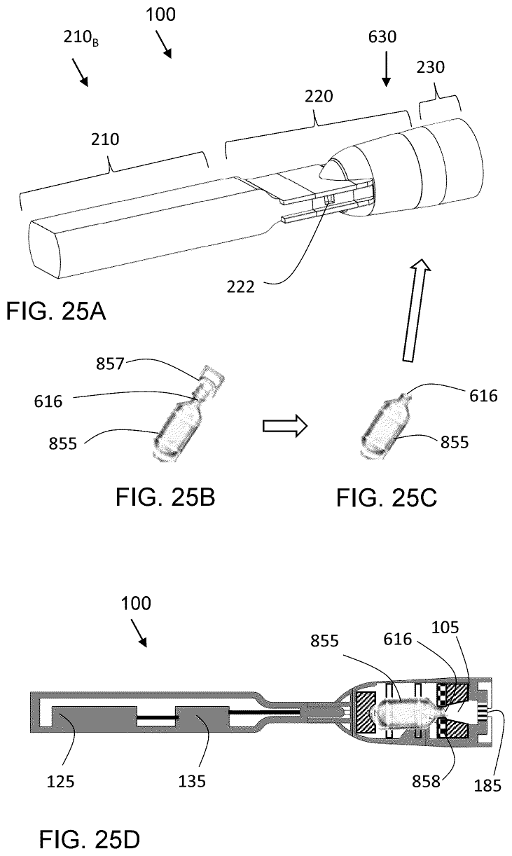

As illustrated in FIGS. 25A, 25B, 25C and 25D, in some embodiments the secondary fluids compartment comprises a vial 855, the vial 855 is characterized by comprising a soft wall. The vial 855 commonly comprises a narrow outlet port 616.

In some embodiments, as illustrated in FIGS. 25B, 25C and 25D, the vial 855 is prefilled with a liquid. When in the non-assembled state, before use, a prefilled vial comprises a detachable liquid tight seal 857 which seals the outlet port 616.

In the process of assembly for use, the prefilled vial 855 is commonly taken out in a sealed configuration state (as illustrated in FIG. 25B) out of a kit package comprising multiple vials. The seal 857 is then removed (as illustrated in FIG. 25C). The open vial 855 is then inserted into attachment with fluids chamber 105 of the main body of the aerosol delivery module 630. In embodiments, a liquid tight connection is formed between the vial 855 outlet port 616 and an inlet orifice 858 of the fluids chamber 105, as illustrated for example in FIG. 25D.

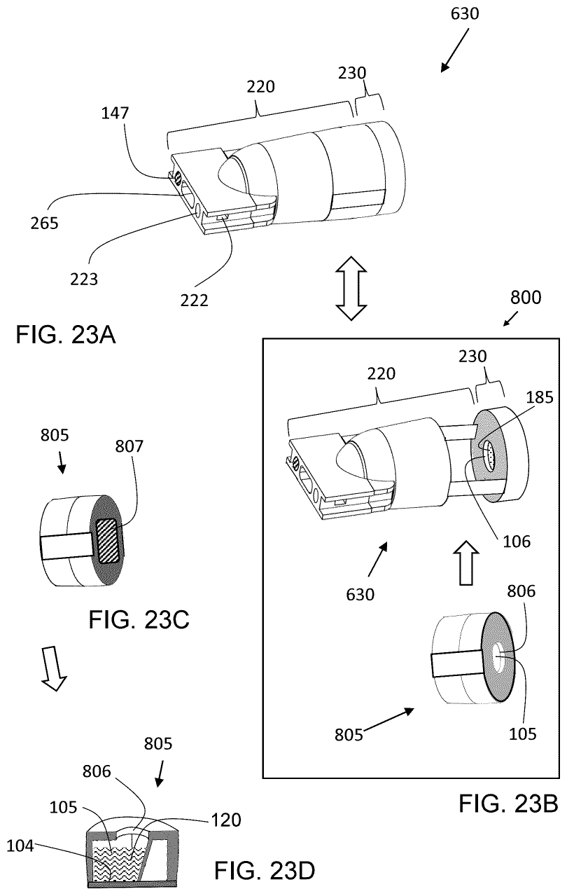

When the quantity of liquid intended for use is small, such as less than 1 ml, or less than 0.5 ml, less than 0.3 ml, there may be preference to have the bulk of the fluids chamber itself constructed as a replaceable cartridge. An example of such an embodiment is illustrated in FIGS. 23A,23B,23C, and 23D. In this embodiment, a portion of the distal fluids compartment 105 comprises an independent fluids cartridge device 805 which is detachably attachable from the full aerosol delivery module 630 comprising the piezo assembly 180.

FIG. 23B illustrates an embodiment wherein the fluids cartridge device 805 is prefilled with a liquid 120. The fluids cartridge device 805 comprises an orifice 806 such that, when in the assembled state attached to the piezo assembly 180, the liquid 120 inside the cartridge device 805 is in fluid communication with the piezo assembly 180 via the orifice 806. In the unassembled state the fluids cartridge device 805 comprises a removable liquid tight seal 807 on the orifice 806.

In the process of assembly for use, the fluids cartridge device 805 is commonly taken out in a sealed configuration state (as illustrated in FIG. 23C) out of a kit package comprising multiple cartridges. The seal 807 is then removed (as illustrated in FIG. 23B). The unsealed fluids cartridge device 805 is then inserted into the main body of the aerosol delivery module 630, thereby forming a complete attachment with fluids chamber 105 in fluid communication with the mesh membrane 185.

In most practical uses, the cartridge device 805 comprise a compartment of volume greater than 0.1 ml and less than 4 ml, or less than 3 ml, or less than 2 ml, or less than 1 ml, or less than 0.5 ml.

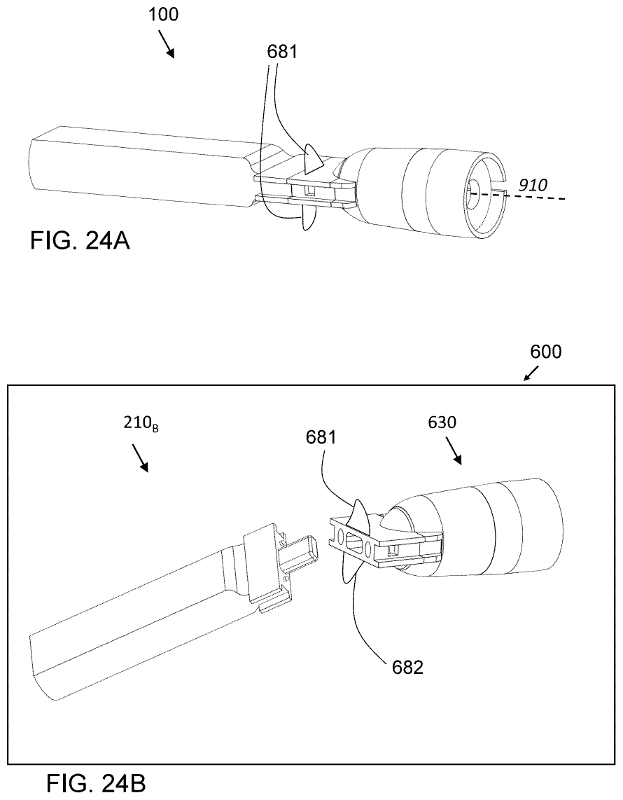

FIGS. 24A and 24B illustrate an embodiment of the system of the present invention comprising a sliding safety feature, which is meant to prevent excessive distal sliding of the device into the oral cavity of the human user. Such a safety feature comprises one or more lateral protrusions 681. For better effectiveness, the height of a protrusion 681 is more than 3 mm and the thickness of the protrusion is less than 5 mm. In embodiments, for better user comfort and to maintain the compactness of the device, the lateral extension of a protrusion is less than 20 mm. The illustrated embodiment shows dual sided protrusions 681. But some embodiments may function well with only one-sided protrusion 861. In some embodiments a protrusion 681 is located at the device mid-section neck portion 220.

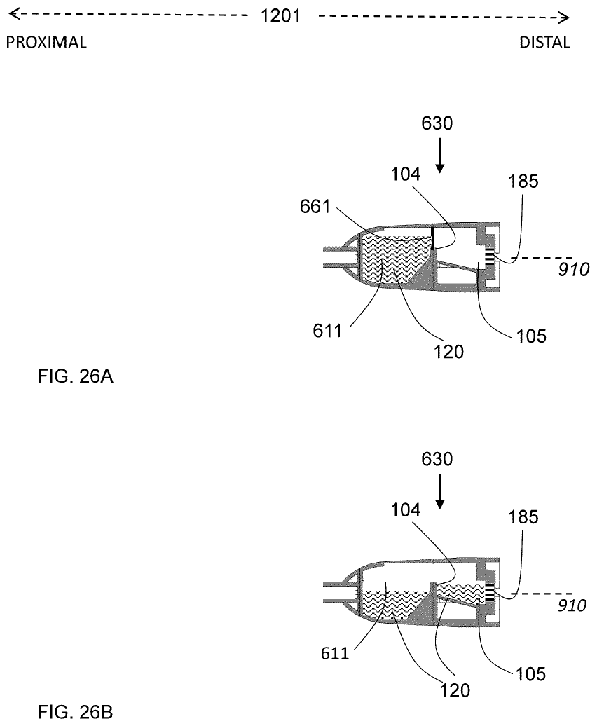

FIGS. 26A and 26B illustrate an embodiment of the system with a prefilled liquid in the secondary fluids compartment 611. In some embodiments of the system, a part of the aerosol delivery module 630 is prefilled with a liquid 120. In some embodiments, a part of the aerosol delivery module 630 is prefilled with a liquid 120 such that the liquid is in contact with the liquid 120 is at least partially filled into the distal fluids compartment 105, thereby also in contact or in fluid communication with the mesh membrane 185. In some embodiments of the system, (i) at a first state the liquid is prefilled and sealed in a secondary fluids compartment 611, i.e., the ‘integral’ compartment 611, (which is an ‘integral’ part of an aerosol delivery module 630), such that it is prevented from flowing into the distal fluids compartment 105, thereby preventing contact of the liquid 120 with the mesh membrane 185, e.g., during initial storage period. For example, as illustrated in FIG. 26A, a barrier 661 prevents liquid flow between the secondary fluids compartment 611 and the distal fluids compartment 105 in a non-operative state, i.e., a state in which the inhalation system 100 is not operating, and, in some embodiments, cannot operate. In other words, barrier 661 impedes flow of the liquid 120 between the secondary fluids compartment 611 and the mesh membrane 185. At a second state, i.e., an operative state in which the inhalation system 100 is operative or can operate once enabled to produce an aerosol, the barrier 661 is at least partly removed or broken or rendered ineffective (e.g., perforated) so as to enable flowing of the liquid from the secondary fluids compartment 611 into the distal fluids compartment 105, thereby enabling contact of the liquid 120 with the mesh membrane 185. In some embodiments, the barrier 661 is recoverable or resealed, such that to prevents liquid flow between the secondary fluids compartment 611 and the distal fluids compartment 105

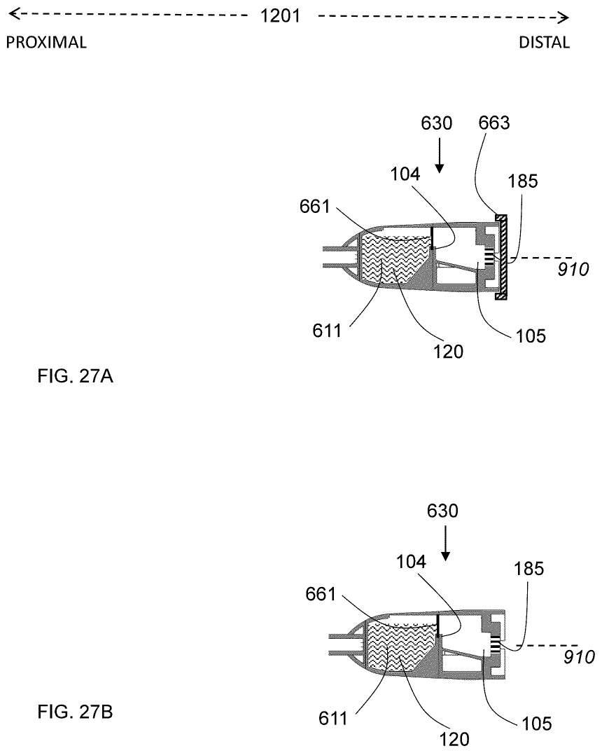

FIGS. 27A and 27B illustrate an embodiment of the system with a distal protection cover. In some embodiments of the system, at an initial state (i) a distal part of the aerosol delivery module 630 is covered with a protection distal cover 663, such that the mesh membrane 185 is protected before use, e.g., during storage, for example as illustrated in FIG. 27A. Before use, at a second state (ii) the protection distal cover 663 is removed, such that the mesh membrane 185 is in unobstructed fluid communication with the ambient air.

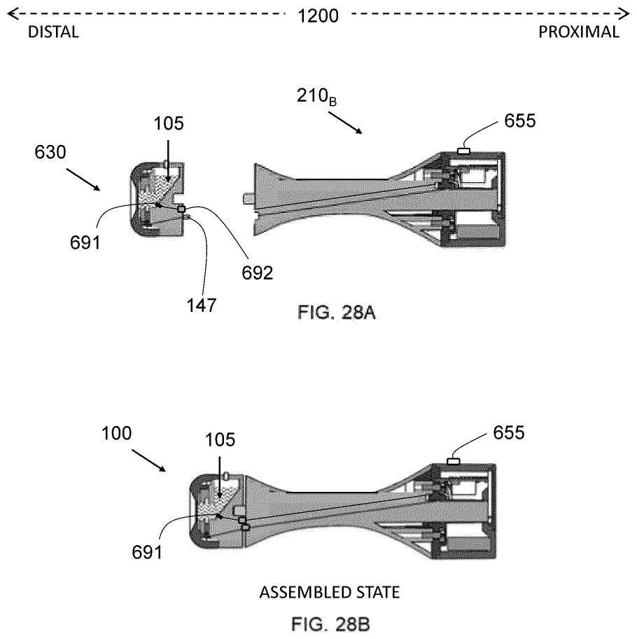

FIGS. 28A and 28B illustrate, in unassembled and assembled states, an embodiment in which the aerosol delivery module 630 includes a sealed distal fluids compartment 105 that is prefilled with a liquid 120. In this embodiment, the aerosol delivery module includes no narrow section (i.e., part of a neck section 220), and the neck portion 220 and substantially all narrow locations are part of the proximal control module 210B.

FIGS. 28A and 28B further illustrate preferred embodiments comprising a fill-level sensor. The fill-level sensor inclusion can be made to be comprised in any of the previously discuss embodiments. Hence, the non-marking of a fill-level sensor in any drawing should not be understood as an indication of absence. The fill-level sensor comprises one or more electrodes 691. In embodiments where the liquids compartment is attachable and/or re-attachable (such as with a liquids compartment cassette, or attachable distal module 630 comprising the liquids compartment), connection contacts 692 may be extending from the internal electrode 691 to the external contact 692. Then when in an assembled state, electronic connection is established between the electronic contact 692 and the control module. The electrodes are embedded in the liquids compartment(s) such that when liquid is filled in the compartment it covers a portion of the electrode. In the assembled state, resistance between the electrodes is measured in communication with the control module circuitry. When the liquid fill level in the compartment gets low enough, the reduces covering by liquid of the electrode leads to increased associated resistance measurement. The increased resistance is then interpreted as a detection of low fill level in the compartment.

In some preferred embodiment, the system is geared for delivery of pre-set and pre-filled small dosages, typically less than 2 mL. Moreover, in such embodiments, it may be preferred to have a single switch turn-ON and automatic turn-OFF functionality. In particular, for such applications a preferred embodiment of the invention is an inhalation delivery system for carrying out a drug inhalation procedure by a human user comprising:
a. an aerosol delivery module comprising a piezo assembly including: (i) an ultrasonically vibrable mesh membrane, (ii) a distal fluids compartment proximal to the mesh membrane in fluid communication with the mesh membrane, and (iii) an aerosol outlet for a mist comprising droplets of the liquid and generated by the mesh membrane; (iv) a distally extended mouthpiece in fluid communication with the mesh membrane and the aerosol outlet, the distal end of the system being the distal end of the mouthpiece;
b. a control module comprising (i) a battery power source, (ii) a control circuitry for electrically powering the piezo assembly, (iii) an inhalation sensor, (iv) an activation switch configured for switching the control module between an initial OFF-state to a subsequent ON-state; and
c. a liquid comprising a drug formulation.

When the inhalation system is in an assembled state such that the aerosol delivery module is attached to the control module and the liquid is contained within the distal fluids compartment in fluid communication with the mesh membrane, the system is configured to have at least two activation states.

When the system is at an OFF-state the control circuitry does not drain power from the battery.

When the system is turned on, for facilitating hands-free dynamic activation, the control circuitry is configured to detect and/or distinguish between at least two inhalation states, (A) a non-inhalation state, and (B) an inhalation event state.

When the activation switch is at an ON-state, the control circuitry is pre-set such that (i) the piezo assembly is powered to vibrate the mesh membrane and emit a mist of the liquid only after the detecting an inhalation event conditioned on a sensor threshold limit (ii) a pre-determined first dose quantity of the liquid is configured for delivery, the first dose quantity is more than 0.1 mL and less than 2 mL.

In preferred embodiments, it is advantageous to both inform and alert the user to the state of the dose delivery. The system is preferably pre-set for a specific default dose. The liquids compartment is preferably comprising at least a portion of the distal liquids compartment bounding walls being of sufficient transparency such that a level of the liquid fill level surface is visually discernable as a change of color or hue. Yet such visual information of the fill level by the user may not be sufficiently precise and/or requires active engagement and attention by the user. In some preferred embodiments, and external indicator, associated with an electronic indicator module, provides external signal of the state of dose delivery. In particular the End-of-dose status is associated with a corresponding indicator state, which may be visual (such as by an LED light) or audible (such as by an associated alarm sound).

The dose quantity itself can be defined and/or controlled in various ways. In one preferred embodiment to define and/or determine the dose quantity is by the target quantity of emitted liquid. The quantity emitted can be assessed and/or defined by a fill-level sensor. The fill level sensor may detect the different between two fill levels of the liquids compartment. A pre-determined fill-level sensing may correspond to a detection that a dose has been reached, or a corresponding End-of-dose event.

In another preferred embodiment to define and/or determine the dose quantity is by a total period or duration of mesh ON-state activation. The logic is that there is an estimated average emission rate of the mist when the mesh membrane vibration is active. Therefore, there is a corresponding liquids emission rate quantity which is estimated to have been delivered during the period.

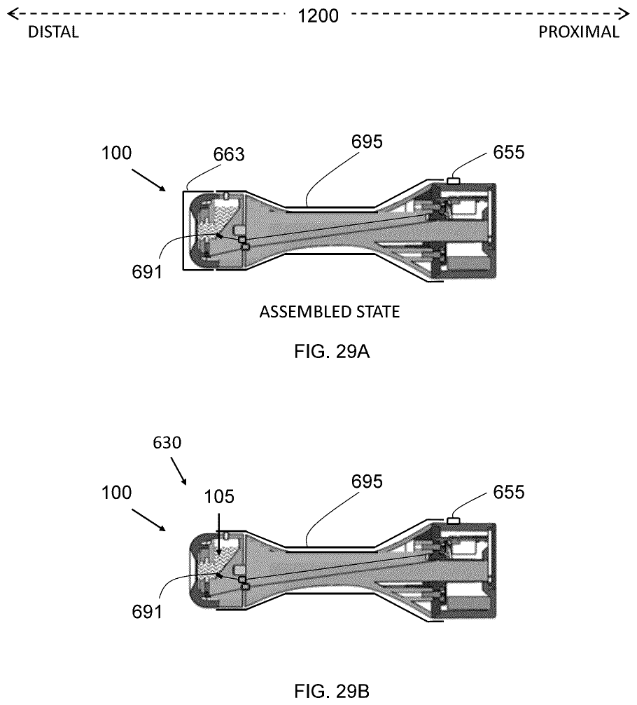

As illustrated in FIGS. 29A and 29B, and as we noted previously, in some preferred embodiments the system may further be comprising of a distal cap 663 or other forms of protective cover 663. For example, a protective cover over at least a portion of the distal tip, such as the tip of the mouthpiece, which also serve to protect the delicate mesh membrane from external damage. Such a cover may also serve as a visual indicator that the system has not been used or tempered with. This is particularly important with medical drug delivery application which are supposed to be single use.

In some preferred embodiments, usability may be simplified and/or streamlined, in constructions where the protective cap or cover also serve as an activation switch. Thereby, the natural act of removing a cover before use, also serve as the step for turning on the system to an ON-state from a previous or packaged initial OFF-state. For example, in FIG. 29A the system is illustrated in an initial assembled state with the protective cap 663 covering over the distal tip of the system, the system is further initially at an OFF-state. The action step of removing of the cap (e.g., by a human user) both exposes the distal tip and simultaneously switches the system electronically to an ON-state. In preferred embodiments the system switching into an ON-state is also having an external indicator module manifestation in a changing state of the indicator 655 (e.g., LED light illumination change).

In some preferred embodiments, an indicator is signaling externally the transition into an ON-state from a previous or packaged initial OFF-state.

In some preferred embodiment, such as when the system is intended for multiple time usage, the cap is reversibly attachable to the mouthpiece. Generally in such embodiments, the cap is a reversible switch such that reversing a movement of the cap enable switching the control module between an initial ON-state to a subsequent OFF-state.

The above discussion of the manipulation of the cap or protective cover by removal should not be considered as limiting. The cap or protective cover manipulation is an activation switch, such that a movement of the cap is switching the control module between an initial OFF-state to a subsequent ON-state, may be different, such as rotating movement or push/pull movement.

For switching, in preferred embodiment, the movement of the cap closes an electric contact or an electric circuit which is previously open.

As illustrated in FIGS. 29A and 29B, in some preferred embodiments the system is further comprising of an optional protective casing or shell 695. The protective casing 695 preferably leaves exposed the switching element (e.g., a cover cap) and an indicator, thereby operation of the system can be performed without removal of the protective casing. The protective casing is preferably rigid.

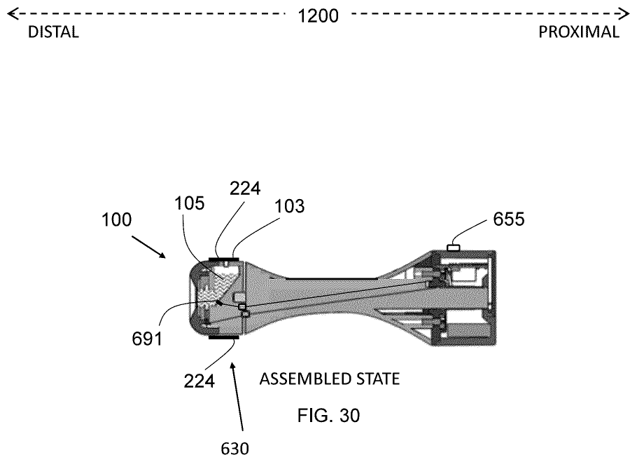

As illustrated in FIG. 30, a taste-producing surface section 224 can be provided in the distal portion 230 in the form of an elastic band. The taste-producing surface can be implemented in the form of a flavoring agent or a scented embedded agent or coating. The elastic band can be reversibly removable, such that the user can place or change between different elastic band. In some preferred embodiments, the elastic band also covers over an inlet or a filling port 103 into the liquids chamber.

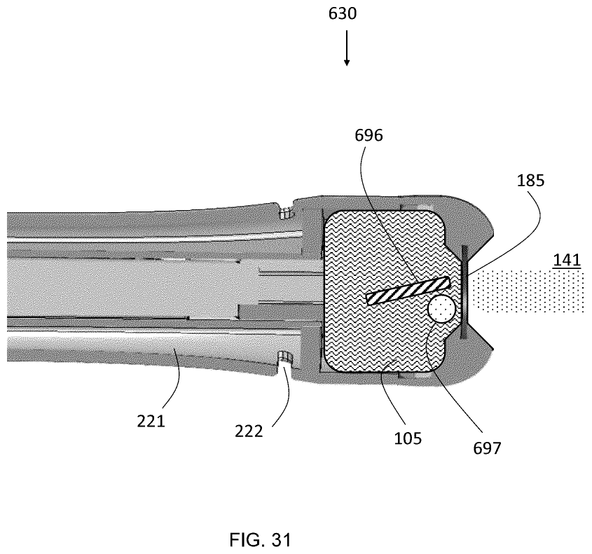

FIG. 31 illustrates a preferred embodiment of the system wherein the aerosol delivery module 630 further comprising a deflecting protrusion or protrusion 696 within the distal fluids compartment 105, wherein the deflecting protrusion 696 extends from a protrusion-distal-end located in proximity to the mesh membrane 185 to a protrusion-proximal-end located at least 2 mm more proximal from the mesh membrane than the protrusion-distal-end. The purpose of the deflecting protrusion is effective to deflect air bubbles 697 preferentially upwards and/or sideways away from the mesh membrane for air bubbles coming into the distal liquids chamber through the mesh membrane when the mesh membrane is powered to vibrate and eject a droplets liquid mist 141 out in the distal direction.

In some preferred embodiments, the deflecting protrusion 696 is a ridge raised from the floor of the distal fluids compartment 105 by more than 1 mm and less than 10 mm. But this is not meant to be limiting. The deflecting protrusion 696 can also be extended from a side or roof portion of the fluids compartment 105.

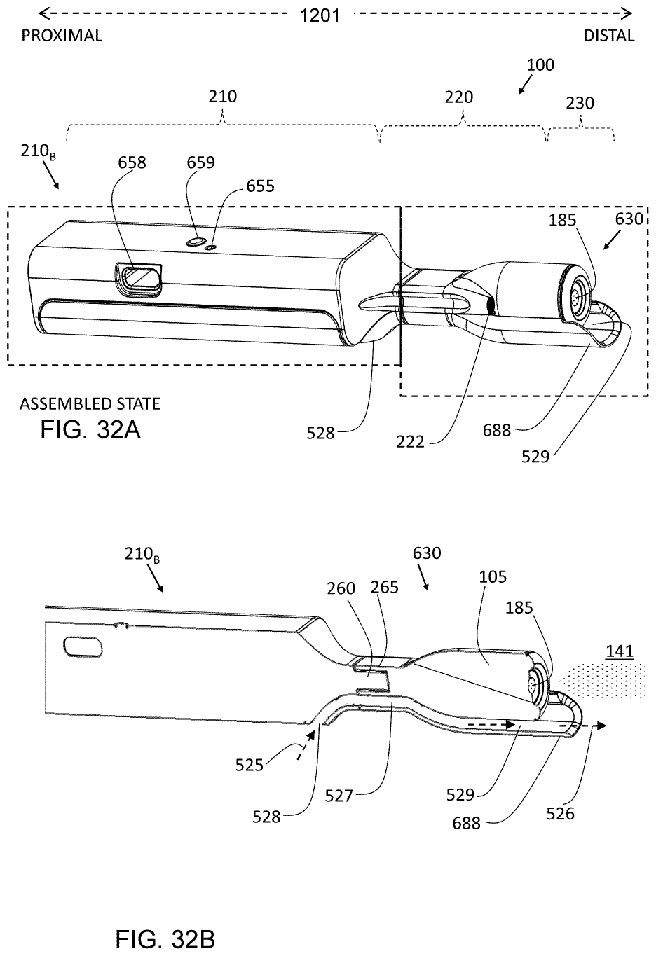

FIGS. 32A and 32B illustrates a preferred embodiment of the system wherein the aerosol delivery module 630 further comprising a tongue-depressing element 688 which extends more distally from the mesh membrane 185 by at least 5 mm and preferably less than 30 mm. The tongue-depressing element 688 extends at a level under the mesh membrane, such that when the device 100 is in use inside the mouth of a human user the tongue-depressing element 688 keeps the tongue under it at a level below the level of the mesh membrane. Thereby, there is reduced possibility that the aerosol ejected 141 from the mesh membrane will collide with and settle onto the tongue tissue.

FIGS. 32A and 32B also illustrates a preferred embodiment of the system wherein the aerosol delivery module 630 further comprising a bottom-air-channel or air conduit element 527. The bottom-air-channel element 527 is comprising a proximal entry port 528, and a distal exit port 529 at a level below the center the mesh membrane. Thereby, an air passage for conduit of air around the mesh membrane at a level below the center the mesh membrane. When the device 100 is positioned in use inside the mouth of a human user, the proximal entry port 528 is situated outside of the mouth and enables ambient entering-air-flow 525 to enter into the bottom-air-channel element 527, and to distally channel an exiting-air-flow 526 via a distal exit port 529 at a level below the center the mesh membrane. The exiting-air-flow 526 provides an added buffering between the ejected aerosol stream 141 and the tongue under it at a level below the level of the mesh membrane. Thereby, there is reduced portion the aerosol ejected 141 from the mesh membrane that collide with and settle onto the tongue tissue, compared when no air flows in the bottom-air-channel element 527 (as can be tested experimentally by blocking of the bottom-air-channel element 527).

The modular construction of the system enables economical packaging of the system into various sets of kits, each with its own advantages and potential use scenarios.

In some embodiments the system is intended for use as a disposable medication container. For example, the prefilled aerosol delivery module 630 is sealed such that external access to the respective fluids compartments 105, 611 is permanently blocked, in order to prevent additional liquid from being refilled in. Such embodiments are useful for sterile medication delivery and to prevent refill of medication by unprofessional end-user patients.

Most of the variability is in the aerosol delivery module 630. Therefore, embodiments of a kit may comprise more than one construction of the aerosol delivery module 630 which are connectable and operable with the same control module 210B that includes a power source 125, and preferably also the sensor 126.

An embodiment of a fully functional starter kit of the invention inhalation system comprises a packaging of the system 100 in a container such that the control module 210B that includes a power source 125 is detached from the aerosol delivery module 630.

The modularity and small size of the fluids cartridges is conductive for the construction of both unfilled and pre-filled cartridges kits. In embodiments, a kit of cartridges comprising one or more secondary fluids compartment(s) such as cartridges 612 and/or vials 855, prefilled with a liquid, may be packaged in a container, wherein a cartridge 612 or vial 855 is designed to fit to connect into a stable assembly in the aerosol delivery modules 630, such that in the assembled state the interior of the cartridge 612 or vial 855 is in fluid communication with the distal fluids compartment 105, e.g., via an outlet port 616.

In some embodiments, a kit comprising one or more cartridge device 805 prefilled with a liquid, is packaged in a container, the cartridge device 805 comprises a removable liquid tight seal 807 on an orifice 806, the cartridge device 805 is designed to fit to connect into a stable assembly in the inhalation system 100 of an aerosol delivery module 630, such that when in the assembled state attached to the piezo assembly 180, the liquid 120 inside the cartridge device 805 is in fluid communication with the piezo assembly 180 via the orifice 806.

The inhalation system of the present invention can be used for aerosol delivery of a variety of liquid 120 substances, primarily for medicinal purposes. Particular examples of such liquids include:
a. an FDA approved drug;
b. a bronchodilator medication, including but not limited to one of albuterol, levalbuterol, ipratropium, aclidinium, arformoterol, formoterol, glycopyrrolate, indacaterol, olodaterol, revefenacin, salmeterol, tiotropium, umeclidinium, Terbutaline, or a mixture thereof;
c. a corticosteroid medication, including but not limited to one of Fluticasone, Budesonide, Prednisolone, or a mixture thereof;
d. a muscarinic medication, including but not limited to one of Revefenacin, Ipratropium bromide, Tiotropium bromide, or a mixture thereof;
e. a Mucoactive medication, including but not limited to one of carbocysteine, erdosteine, N-acetylcysteine, or a mixture thereof;
f. a anti-inflammatory medication, including but not limited to Methylxanthines;
g. nicotine;
h. an antimicrobial agent, including but not limited to one of Silver nanoparticles, PVP Iodine, tobramycin, colistin, and aztreonam lysine;
i. a vaccine. Of particular interest and potential effective advantage is for a vaccine for a respiratory infection caused by a virus selected from influenza or corona viruses;
j. a cannabinoid substance;
k. vitamin B12
l. a liposomes-encapsulated, pharmaceutically-active substance.

For the liquid to be suitable or optimized for use with the system of the present invention, some preferred properties are advantageous to pass the liquid through the mesh membrane. In particular where any one or a combination of these properties is satisfied for the liquid 120:
a. the liquid 120 comprises an aqueous in the sense that more than 50% of the liquid composition is water;
b. the liquid 120 comprises an aqueous emulsion;
c. the emulsion comprises droplets having a size distribution peak at droplets size larger than 5 nm and smaller than 100 nm;
d. the emulsion comprises droplets having a size distribution peak at droplets size larger than 10 nm and smaller than 90 nm;
e. the emulsion comprises droplets having a size distribution peak at droplets size larger than 20 nm and smaller than 80 nm;
f. the emulsion liquid has surface tension such that the contact angle of a 3 mm diameter droplet of the liquid with mesh membrane is less than 90 degrees;
g. the liquid 120 comprises an aqueous colloid of nanoparticles, wherein the nanoparticles have a zeta potential of size greater than 10 mV;
h. the nanoparticles diameter is having a size distribution peak at size larger than 2 nm and smaller than 100 nm.

In some embodiments, the ultrasonic mesh nebulizer of the present invention is implemented to emulate the use targets of a pressurized metered dose inhaler (pMDI). A typical pMDI implements a pressurized propellant gas to spray a short burst of dose of liquid droplets, typically of total liquid droplets spray volume, total dose or interchangeably target dose (TD) between 0.05 ml and 0.2 ml. Most commonly, TD is around 0.12 ml, such as between 0.08 ml to 0.12 ml. For example, such pMDI are use for Asthma treatment with Ventolin.

In some embodiments of the present invention, a method of emulating meter-dose-inhaler (MDI) dosage with an ultrasonic nebulizer is provided. Our ultrasonic mesh nebulizer, is characterized by
a. an electronic circuit which is
   i. having a predetermined average rate of R1 milliliter per minute (ml/min) aerosol emission rate;
   ii. comprising a pressure sensor connected to an electronic circuit;
b. activating the mesh ultrasonic vibration for a duration Di by the electronic circuit at each time Ti when the sensor detects a pressure difference below a pre-set thresh-hold limit L1, thereby a fractional dose F(Di,Ti)=Di*R1 milliliter;
c. Indicating when the cumulated dose of the sum of at least a selection of previous F(Di,Ti) is greater than a pre-set target dose TD.
   Thereby, the user knows that the target dose TD was reached.

### Additional Discussion

1. An inhalation system for delivery of an aerosol to the oropharynx of a human user, the inhalation system comprising:
   a. an aerosol delivery module comprising a piezo assembly including: an ultrasonically vibrable mesh membrane, a distal fluids compartment in fluid communication with the mesh membrane, and an aerosol outlet for a mist comprising droplets of the liquid generated by the mesh membrane; and
   b. a control module,
   wherein when the inhalation system is in an assembled state such that the aerosol delivery module is distally disposed and/or the control module is attached thereto, the assembled inhalation system is shaped to include a narrow section characterized by the following:
      a minimum cross-sectional dimension of the narrow section is at least 10% smaller than a minimum cross-sectional dimension of the aerosol delivery module at the mesh membrane, and/or
      when the user's lips and/or teeth are transversely engaged with the narrow section, the mesh membrane resides within the user's oral cavity and the aerosol outlet is in direct fluid communication with the user's oropharynx.
2. An inhalation system for delivery of an aerosol to the oropharynx of a human user, the inhalation system comprising:
   a. an aerosol delivery module comprising a piezo assembly including: an ultrasonically vibrable mesh membrane, a distal fluids compartment in fluid communication with the mesh membrane, and an aerosol outlet for a mist comprising droplets of the liquid generated by the mesh membrane;
   b. a control module;
   c. a bottom-air-channel or air conduit element 527, the bottom-air channel is passing along a path that is passing around a side of the mesh membrane from a more proximal to a more distal side of the mesh membrane, at a level at least partially or predominantly below the center the mesh membrane.
   Wherein, when the inhalation system is in an assembled state, the aerosol delivery module is attached to and/or is predominantly distally disposed thereto the control module.
3. The previous inventive concept wherein when the inhalation system is in an assembled state, the assembled inhalation system is shaped to include a narrow section characterized by the following:
   a minimum cross-sectional dimension of the narrow section is at least 10% smaller than a minimum cross-sectional dimension of the aerosol delivery module at the mesh membrane, and/or
   when the user's lips and/or teeth are transversely engaged with the narrow section, the mesh membrane resides within the user's oral cavity and the aerosol outlet is in direct fluid communication with the user's oropharynx.
253. An inhalation system for delivery of an aerosol to the oropharynx of a human user, the inhalation system comprising:
   a. a distal aerosol delivery module 630 comprising a device distal portion 230 of the system distal module 630 including (i) an aerosol outlet 186 defining a mist-exiting location and (ii) a piezo assembly 180 including an ultrasonically vibrable mesh membrane, which upon electrical activation produces a mist comprising droplets of the liquid, the mesh membrane 185 defining a mist-generating location;
   b. the distal module 630 further comprising a distal fluids compartment 105 in fluid communication with the mesh membrane 185; the compartment 105 comprising a compartment-proximal-wall 104, and
   c. a device mid-section neck portion 220 including a narrow section, the narrow section being characterized by having a location of a narrow width cross-sectional dimension WN that is at least 10% smaller than a minimum width WD dimension of a more distal cross-section at a membrane plane 900 passing through the mesh membrane, the membrane plane 900 being perpendicular to the geometrical axis 910 perpendicular to and passing through the center of the mesh membrane 185;
   wherein the inhalation system is shaped such that when the user's lips and/or teeth are transversely engaged with the narrow section, the mist-generating location 185 resides within the user's oral cavity and the mist-exiting location is in direct fluid communication with the user's oropharynx.
254. The previous inventive concept wherein at least part of the neck portion 220 is within the distal module 630 connected to the device distal portion 230.
255. The inhalation system of inventive concept 253 having an elongated shape such that the geometrical axis 910, perpendicular to and passing through the center of the mesh membrane 185, defines a longitudinal axis of the system in the sense that the dimensional extension of the system along the longitudinal a longitudinal-axis oriented similar to the axis 910 is larger than the dimensional extension of the system along any axis perpendicular to the axis said longitudinal axis, wherein said longitudinal-axis is passing through the center of the mesh membrane 185 and is at an angle deviating less than 30 degrees from the axis 910.
256. The inhalation system of inventive concept 253 wherein the narrow section is characterized by having a location of a narrow width cross-sectional dimension WN that is at least 20% smaller, or at least 30% smaller, or at least 50% smaller, or at least 70% smaller, than a minimum width WD dimension of a more distal cross-section at a plane 900 passing through and tangential to the center of the mesh membrane.
257. The previous inventive concept wherein narrow width cross-sectional dimension WN is of size between 8 mm and 10 mm, or between 6 mm and 8 mm, or between 4 mm and 6 mm, or less than 4 mm.
258. The inhalation system of inventive concept 253 wherein the mesh membrane 185 is located in proximity to the distal most edge 191 of the system 100 of less than 1 mm, or less than 8 mm, or less than 5 mm, or less than 3 mm.
259. The inhalation system of inventive concept 253 wherein the distal fluids compartment 105 is bounded by a compartment-proximal-wall 104 which is proximal to the mesh membrane 185, such that the compartment-proximal-wall 104 is extended above and below a height of the line 900 perpendicular to the middle of the of the mesh membrane 185.
260. Inventive concept 253 wherein the fluids compartment 105 has a volume of more than 0.1 ml and less than 5 ml, or less than 4 ml, or less than 3 ml, or less than 2 ml, or less than 1 ml.
261. Inventive concept 253 wherein the fluids compartment 105 is in fluid communication with a filling port 103.
262. The previous inventive concept wherein the filling port 103 is comprising a check-valve.
263. The inhalation system of inventive concept 253 wherein a center of gravity of the inhalation device is being displaced proximally more than 3 cm from a mesh membrane 185 when the inhalation device is in a liquid-empty state.
264. The inhalation system of inventive concept 253, additionally comprising a proximal device portion 210 that is part of the base device portion 210B which is characterized by comprising a power source 125 and/or a control circuitry 135, for electrically powering the piezo assembly 180.
265. The previous inventive concept wherein the base device portion 210B is proximal to the distal portion 230, such that more than 50% of the mass of the power source is proximal to the mesh membrane 185.
266. The previous inventive concept wherein proximal device portion 210 is proximal to the neck portion 220.
267. The previous inventive concept wherein at least part of the neck portion 220 is within the distal module 630 connected to the device distal portion 230.
268. Any of the previous inventive concepts wherein the base device portion 210B is attachable and detachable from the distal section 230.
269. The previous inventive concept, wherein a proximal mechanical or magnetic attachment element 260 of the base device portion 210B is linked to a distal mechanical or magnetic attachment element 265 of the mid-section 220.
270. Inventive concept 265 wherein distal electrical contacts 147 are electrically connected to the piezo assembly 180, and where proximal electrical contacts 148 are connected to the power source 125, and wherein in the assembled state of the system the proximal electrical contacts 148 are engaged with the distal electrical contacts 147, establishing an electrical communication between the piezo assembly 180 and the power source 125 and/or the electronic control circuit 135.
271. The inhalation system of any one of inventive concepts 253, additionally comprising an inhalation sensor 126.
272. The previous inventive concept, wherein the inhalation sensor 126 is in fluid communication with a distal port 222, the inhalation sensor 126 is effective to detect an air pressure difference between an air pressure at the distal port 222 and an ambient air pressure more proximal than the distal port 222.
273. The previous inventive concept, comprising control circuitry 135 configured to initiate and/or cease activation of the piezo assembly 180 in response to detection of an air pressure difference greater than a threshold limit, and/or cease activation of the piezo assembly 180 in response to detection of an air pressure difference less than a pressure threshold limit.
274. The previous inventive concept wherein the threshold limit is a pressure between 2 cm H2O and 20 cm H2O.
275. The previous inventive concept wherein the threshold limit is a pressure between 2 cm H2O and 15 cm H2O.
276. The previous inventive concept wherein the threshold limit is a pressure between 5 cm H2O and 15 cm H2O
277. The previous inventive concept, wherein the distal port 222 is located distally from the inhalation sensor 126.
278. Any of the previous inventive concepts wherein the inhalation sensor 126 is located in the base device portion 210B.
279. The previous inventive concept wherein the system in the assembled state comprises a fluid communication between the distal port 222 to the inhalation sensor 126 via a lumen 221, and wherein in the unassembled state a distal portion 223 of the lumen 221 is within the mid-section of the device, and a portion 225 of the lumen 221 is within the base device portion 210B.
280. The inhalation system of inventive concept 253, further comprising an extension fluids chamber 610 in fluid communication with the distal fluids compartment 105.
281. The previous inventive concept wherein the extension fluids chamber 610 is an independent cartridge device detachably attachable to the distal fluids compartment 105.
282. The previous inventive concept wherein the extension fluids chamber 610 is prefilled with a liquid 120.
283. The previous inventive concept wherein the volume of extension fluids chamber 610 is larger than the volume of the distal fluids compartment 105.
284. The previous inventive concept wherein extension fluids chamber 610 comprise an outlet port 616, such that in the assembled state the interior of the extension fluids chamber 610 is in fluid communication with the distal fluids compartment 105 via outlet port 616.
285. The previous inventive concept wherein a width of the aperture of outlet port 616 across the center of the aperture is larger than 2 mm and less than 5 mm.
286. Any of the previous inventive concepts wherein the extension fluids chamber 610 comprises a vial 855, the vial 855 comprising a soft wall, and the vial 855 comprising an outlet port 616.
287. The previous inventive concept wherein the vial 855, when in the non-assembled state, comprises a detachable liquid tight seal 857 which seals the outlet port 616.
288. The previous inventive concept wherein the vial 855 is pre-filled with a liquid.
289. The inhalation system of inventive concept 253, wherein a portion of the distal fluids compartment 105 comprises an independent fluids cartridge device 805 which is detachably attachable from the piezo assembly 180.
290. The previous inventive concept wherein the fluids cartridge device 805 is prefilled with a liquid 120.
291. The previous inventive concept wherein the fluids cartridge device 805 comprises an orifice 806 such that, when in the assembled state attached to the piezo assembly 180, the liquid 120 inside the cartridge device 805 is in fluid communication with the piezo assembly 180 via the orifice 806.
292. The previous inventive concept wherein in the unassembled state the fluids cartridge device 805 comprises a removable liquid tight seal 807 on the orifice 806.
293. Any of the previous inventive concepts wherein the cartridge device 805 comprise a compartment of volume greater than 0.1 ml and less than 4 ml, or less than 3 ml, or less than 2 ml, or less than 1 ml, or less than 0.5 ml.
294. The inhalation system of any of the previous inventive concepts, wherein when compartment 105 is filled with an amount of liquid 120 less than 0.5 ml and the system 100 is oriented horizontally, such that the axis 910 is perpendicular to the force of gravity, the average surface of the liquid 120 is at a level higher than the middle of the mesh 185.
295. The inhalation system of any of the previous inventive concepts, wherein when compartment 105 is filled with an amount of liquid 120 less than 0.2 ml and the system 100 is oriented horizontally, such that the axis 910 is perpendicular to the force of gravity, the average surface of the liquid 120 is at a level higher than the middle of the mesh 185.
296. Any of the previous two inventive concepts inventive concept, wherein in a second orientation, such that the axis 910 is more than 30 degrees below horizontal, the liquid 120 is in contact with the mesh membrane 185, and the average surface of the liquid 120 at a level higher than 2 mm below the center of the mesh 185.
297. The inhalation system of inventive concept 253, wherein the device distal module 630 is shaped such that when the user's lips and/or teeth are transversely engaged with a location of a narrow section, the depth DM of mist-generating location 185 beyond the teeth into the oral cavity is at least 1 cm deep, or at least 2 cm deep, or at least 3 cm deep.
298. The previous inventive concept wherein the location of the port 222 is deeper into the oral cavity than the external edge 15L or 15U of the lips.
299. Any of the previous inventive concepts wherein the inhalation system further comprising one or more protrusions 681.
300. The previous inventive concept wherein the height of a protrusion 681 is more than 3 mm and the thickness of the protrusion is less than 5 mm.
301. The previous inventive concept wherein a protrusion 681 is located at the device mid-section neck portion 220.
302. A kit comprising more than one construction of the distal module 630 which are connectable and operable with the same base device portion 210B that includes a power source 125, where the constructions of the distal module 630 are of any one of inventive concepts 253 to 300.
303. A kit comprising the inhalation system of any one of inventive concepts 253 to 300, packaged in a container such that the base device portion 210B that includes a power source 125 is detached from the distal module 630.
304. A kit of cartridges comprising one or more extension fluids chamber(s) 610 prefilled with a liquid, packaged in a container, wherein an extension fluids chamber 610 is designed to fit to connect into a stable assembly in the inhalation system of any one of inventive concepts 253 to 300 such that in the assembled state the interior of the extension fluids chamber 610 is in fluid communication with the distal fluids compartment 105 via outlet port 616.
305. A kit comprising one or more cartridge device 805 prefilled with a liquid, packaged in a container, the cartridge device 805 comprises a removable liquid tight seal 807 on an orifice 806, the cartridge device 805 is designed to fit to connect into a stable assembly in the inhalation system 100 of any one of inventive concepts 253 to 300 such that when in the assembled state attached to the piezo assembly 180, the liquid 120 inside the cartridge device 805 is in fluid communication with the piezo assembly 180 via the orifice 806.
306. Any of the previous inventive concepts wherein a part of the distal device portion module 630 is prefilled with a liquid 120.
307. The previous inventive concept wherein a part of the distal device portion module 630 is prefilled with a liquid 120 and the liquid 120 in fluid communication with the mesh membrane 185.
308. Inventive concept 306 wherein, (i) at a first state the liquid is prefilled and sealed in an extension fluids chamber 610 such that a barrier 661 impedes flow of the liquid 120 between the fluids chamber 610 and the mesh membrane 185.
309. The previous inventive concept wherein, (ii) at a second state, to enable active aerosol production use, the barrier 661 is removed or broken and fluid communication of the liquid 120 is established from fluids chamber 610 into the distal fluids compartment 105 and the mesh membrane 185.
310. The previous inventive concept wherein the barrier 661 is recoverable or resealable, such that to liquid flow between the chamber 610 and chamber 105 is impeded or blocked.
311. Any of the previous inventive concepts wherein at a first configuration state (i) a distal part of the distal device portion module 630 is covered with a protection distal cover 663, such that the mesh membrane 185 is protected before use.
312. The previous inventive concept wherein at a second configuration state (ii) the protection distal cover 663 is removed, such that the mesh membrane 185 is in unobstructed fluid communication with the ambient air.
313. A prefilled aerosol delivery module 630 comprising:
   a. a distal ultrasonically vibrable mesh membrane 185 in contact with a piezo assembly 180;
   b. electrical contacts 147 which are electrically connected to the piezo assembly 180;
   c. a distal fluid chamber 105 in fluid communication with the mesh membrane 185;
   d. a container portion defining an extension fluid chamber 610; and
   e. a sterile liquid 120 prefilled into the extension fluid chamber 610.
314. The previous inventive concept wherein the liquid 120 in fluid communication with the mesh membrane 185.
315. The prefilled aerosol delivery module 630 of inventive concept 313 wherein, (i) at a first configuration state the liquid is prefilled and sealed in an extension fluids chamber 610 such that a barrier 661 impedes flow of the liquid 120 between the fluids chamber 610 and the mesh membrane 185.
316. The previous inventive concept wherein, (ii) at a second configuration state, to enable active aerosol production use, the barrier 661 is removed or broken and fluid communication of the liquid 120 is established from fluids chamber 610 into the distal fluids compartment 105 and the mesh membrane 185.
317. The previous inventive concept wherein the barrier 661 is recoverable or resealable, such that to liquid flow between the extension fluids chamber 610 and chamber 105 is impeded or blocked.
318. The prefilled aerosol delivery module 630 of inventive concept 313 wherein, at a first configuration state (i) a distal part of the distal device portion module 630 is covered with a protection distal cover 663, such that the mesh membrane 185 is protected before use.
319. The previous inventive concept wherein at a second configuration state (ii) the protection distal cover 663 is removed, such that the mesh membrane 185 is in unobstructed fluid communication with the ambient air.
320. The prefilled aerosol delivery module 630 of inventive concept 313 wherein, external access to the fluid chambers 105 and 610 is permanently blocked such that additional liquid cannot be refilled in.
321. The prefilled aerosol delivery module 630 of inventive concept 313 wherein the combined volume of the fluid chambers 105 and 610 is less than 4 ml, or less than 3 ml, or less than 2 ml, and more than 0.1 ml or more than 0.5 ml.
322. The prefilled aerosol delivery module 630 of inventive concept 313 further comprising a sensor of the liquid fill level.
323. The prefilled aerosol delivery module 630 of inventive concept 313 further comprising an RFID containing identification signals on the liquid 120 which is prefilled.
324. A sterilization secured prefilled aerosol delivery module 630 comprising:
   a. the prefilled aerosol delivery module 630 of inventive concept 313;
   b. a sealed packaging encasing the prefilled module 630;
   such that the prefilled module 630 cannot be taken out of the packaging without breaking the seal.
325. A kit comprising:
   a. the prefilled aerosol delivery module 630 of inventive concept 313;
   b. a power module base device portion 210B which is characterized by (i) comprising a power source 125 for electrically powering the piezo assembly 180, (ii) an electronic control circuitry 135, (iii) base electrical contacts 148;
   the kit elements are configured to be attachable and detachable such that the base electrical contacts 148 are engaged and disengaged with the distal electrical contacts 147, to establish an electrical communication between the piezo assembly 180 and the power source 125 and/or the electronic control circuit 135.
326. The previous inventive concept wherein, (i) The prefilled aerosol delivery module 630 of inventive concept 100 further comprising an RFID containing identification signals on the liquid 120 which is prefilled, (ii) electronic control circuitry 135, comprise means for interacting with the RFID.
327. Any of the previous inventive concepts wherein the system is further comprising an electronic external indicator module 655 in electronic communication with the electronic control circuitry 135.
328. The previous inventive concept wherein the indicator module 655 comprises an LED light.
329. Inventive concept 327 wherein the indicator module 655 comprises a display screen 155.
330. Inventive concept 327 wherein the electronic control circuitry 135 is configured to change the indicator module 655 display state when the piezo assembly 180 is activated.
331. Any of the previous inventive concepts wherein the minimum width WD dimension of a more distal cross-section, at a membrane plane 900 passing through the mesh membrane, is less than 20 mm, or less than 15 mm, or less than 12 mm, or less than 10 mm.
332. A method of assembly of an inhalation system for delivery of an aerosol to the oropharynx of a human user, the inhalation system comprising the steps of:
   a. providing a device distal portion 230 of the system distal module 630 including (i) an aerosol outlet 186 defining a mist-exiting location and (ii) a piezo assembly 180 including an ultrasonically vibrable mesh membrane;
   b. providing a distal fluids compartment 105 in fluid communication with the mesh membrane 185; and
   c. providing a distal sub-assembly module 630 comprising of a device mid-section neck portion 220 connected to the device distal portion 230; the sub-assembly module 630 is further comprising (i) a distal port 222, (ii) an attachment element 265, and (iii) distal electrical contacts 147 electrically connected to the piezo assembly 180;
   d. providing a base device portion 210B that includes a power source 125, the proximal device portion 210 further comprising (i) a pressure sensor 126, (ii) an attachment element 260, and (iii) proximal electrical contacts 148;
   connecting the base device portion 210B to the distal sub-assembly module 630, such that in the assembled state
      (i) there is fluid communication between the distal port 222 to the pressure sensor 126 via a lumen 221;
      (ii) a proximal element 260 of the base device portion 210B is linked to a distal mechanical or magnetic attachment element 265 of the sub-assembly module 630 by a mechanical or magnetic attachment force;
      (iii) the proximal electrical contacts 148 are engaged with the distal electrical contacts 147, establishing an electrical communication between the piezo assembly 180 and the power source 125.
333. The previous method of assembly of an inhalation system of inventive concept 332 wherein the inhalation system is shaped such that when the user's lips and/or teeth are transversely engaged with the narrow section, the mist-generating location 185 resides within the user's oral cavity and the mist-exiting location is in direct fluid communication with the user's oropharynx.
334. The method of assembly of inventive concept of an inhalation system of inventive concept 119 wherein the distal sub-assembly module 630 is constructed such that a mid-section neck portion 220 is including a narrow section, the narrow section being characterized by having a location of a narrow width cross-sectional dimension WN that is at least 10% smaller than a minimum width WD dimension of a more distal cross-section at a membrane plane 900 passing through the mesh membrane, the membrane plane 900 being perpendicular to the geometrical axis 910 perpendicular to and passing through the center of the mesh membrane 185.
335. The method of assembly of an inhalation system of inventive concept 332 further comprising the step of aligning the base device portion 210B and the distal sub-assembly module 630 are such that the geometrical axis 910 perpendicular to and passing through the center of the mesh membrane 185 is also passing through the majority of the length of the distal sub-assembly module 630.
336. The method of assembly of an inhalation system of inventive concept 332 wherein in the assembled state the base device portion 210B and the distal sub-assembly module 630 are aligned such that the resulting inhalation system 100 is having an elongated shape where the geometrical axis 910, perpendicular to and passing through the center of the mesh membrane 185, defines a longitudinal axis of the system in the sense that the dimensional extension of the system along the longitudinal axis 910 is larger than the dimensional extension of the system along any axis perpendicular to the axis 910.
337. The method of assembly of an inhalation system of inventive concept 332 further comprising the step of filling the distal fluids compartment 105 with a liquid 120.
338. The method of assembly of an inhalation system of inventive concept 332 further comprising the step of filling the extension fluids chamber 610 with a liquid 120.
339. The previous inventive concept wherein the liquid 120 is blocked from liquid flow communication with the mesh membrane 185.
340. The method of assembly of an inhalation system of inventive concept 332 further comprising the step of connecting an extension fluids chamber 610 into a stable assembly with the distal module 630, such the extension fluids chamber 610 is in fluid communication with the distal fluids compartment 105 via outlet port 616.
341. The previous inventive concept further comprising the step of detaching a liquid tight seal 857, which seals the outlet port 616, prior to connecting an extension fluids chamber 610 into a stable assembly with the distal module 630.
342. The previous inventive concept wherein the extension fluid chamber 610 is a cartridge pre-filled with a liquid 120.
343. The previous inventive concepts wherein the extension fluids chamber 610 comprises a vial 855, the vial 855 comprising a soft wall, and the vial 855 comprising an outlet port 616.
344. The previous inventive concept further comprising the step of pressing on a soft wall of the vial 855, thereby transferring a portion of the pre-filled liquid 120 out of the vial 855 and into the fluids chamber 105 and bringing the liquid into contact with the mesh 185.
345. The method of assembly of the inhalation system of inventive concept 332, further comprising the step of connecting a pre-filled cartridge device 805 in fluid communication with the piezo assembly 180 via the orifice 806 of the cartridge device 805, wherein the fluids cartridge device 805 is prefilled with a liquid 120.
346. The previous inventive concept further comprising the step of removing a liquid tight seal 807 from covering on the orifice 806.
347. The previous inventive concept wherein the step of removing a liquid tight seal 807 from covering on the orifice 806 is performed prior to the step of connecting a pre-filled cartridge device 805 in fluid communication with the piezo assembly 180.
348. Inventive concept 346 wherein the step of removing a liquid tight seal 807 from covering on the orifice 806 is occurring in the course of the motion of the step of connecting a pre-filled cartridge device 805 in fluid communication with the piezo assembly 180.
349. A method of controlling the activation of an inhalation system for delivery of an aerosol to the oropharynx of a human user, comprising the steps of:
   a. providing an inhalation system 100 comprising a system distal module 630 including (i) an aerosol outlet 186 defining a mist-exiting location; (ii) a piezo assembly 180 including an ultrasonically vibrable mesh membrane; (iii) a distal fluids compartment 105 in fluid communication with the mesh membrane 185; (iv) a power source 125 and an electronic control circuit electrically connected to the piezo assembly 180 and comprising a pressure sensor 126; the inhalation system 100 is further comprising (v) a distal port 222 in fluid communication with the pressure sensor 126 via a lumen 221;
   b. placing the inhalation system 100 such that the mesh membrane 185 is within the oral cavity of the human user at a location more distal than the front teeth;
   c. activating the piezo assembly vibration by generating a low pressure in the neighborhood the distal port 222, the low pressure is lower than ambient air pressure by a threshold difference;
   d. deactivating piezo assembly vibration by generating a pressure in the neighborhood the distal port 222 that is close ambient air pressure within a difference than is less than a threshold difference.
350. The previous inventive concept wherein the threshold of pressure difference is less than 10 cmH2O, or less than 5 cmH2O, or less than 3 cmH2O.
351. The previous inventive concept wherein the threshold of pressure difference is more than 1 cmH2O.
352. A method of emulating meter-dose-inhaler (MDI) dosage with an ultrasonic nebulizer, comprising the steps of,
   a. Providing an ultrasonic mesh nebulizer, the ultrasonic mesh nebulizer is
      i. comprising a distal aerosol delivery module 630;
      ii. having a predetermined average rate of R1 milliliter per minute (ml/min) aerosol emission rate;
      iii. comprising a pressure sensor connected to an electronic circuit;
   b. activating the mesh ultrasonic vibration for a duration Di by the electronic circuit at each time Ti when the sensor detects a pressure difference below a pre-set thresh-hold limit L1, thereby a fractional dose F(Di,Ti)=Di*R1 milliliter;
   c. Indicating when the cumulated dose of the sum of at least a selection of previous F(Di,Ti) is greater than a pre-set target dose TD.
353. The previous inventive concept wherein the target dose TD is greater than 0.05 ml and smaller than 0.5 ml.
354. The previous inventive concept wherein the target dose TD is greater than 0.08 ml.
355. Inventive concept 353 wherein the target dose TD is smaller than 0.3 ml.
356. The previous inventive concept wherein the target dose TD is smaller than 0.2 ml.
357. The previous inventive concept wherein the target dose TD is smaller than 0.12 ml.
358. The method of inventive concept 352 wherein the average rate of R1 is greater than 0.2 ml/min and less than 3 ml/min.
359. The previous inventive concept wherein R1 is greater than 0.5 ml/min.
360. The previous inventive concept wherein R1 is less than 2 ml/min.
361. The previous inventive concept wherein R1 is less than 1 ml/min.
362. The method of any of the previous inventive concepts comprising the distal aerosol delivery module 630 of any of the inventive concepts 40 to 119.
363. Any of the previous inventive concepts wherein the electronic control circuit 135 is configured to controllably produce more than one emission-rate-modes, where each emission rate mode is characterized by an associated average rate per second of aerosol emission by the vibrable mesh 185.
364. The previous inventive concept wherein during a period of one second there is an activation-duration portion of A % during which aerosol is produced, and a non-activation duration portion N % during which aerosol is not produced, where A %+N %=100%, such that at least one emission-rate-mode has a higher activation-duration portion of A %=A1% than a second emission-rate-mode having an activation-duration portion of A %=A2%, such that A1% is greater than A2%.
365. Any of the previous inventive concepts wherein the liquid 120 is comprising an FDA approved drug.
366. Any of the previous inventive concepts wherein the liquid 120 is comprising a bronchodilator medication, including but not limited to one of albuterol, levalbuterol, ipratropium, aclidinium, arformoterol, formoterol, glycopyrrolate, indacaterol, olodaterol, revefenacin, salmeterol, tiotropium, umeclidinium, Terbutaline, or a mixture thereof.
367. Any of the previous inventive concepts wherein the liquid 120 is comprising a corticosteroid medication, including but not limited to one of Fluticasone, Budesonide, Prednisolone, or a mixture thereof.
368. Any of the previous inventive concepts wherein the liquid 120 is comprising a muscarinic medication, including but not limited to one of Revefenacin, Ipratropium bromide, Tiotropium bromide, or a mixture thereof.
369. Any of the previous inventive concepts wherein the liquid 120 is comprising a Mucoactive medication, including but not limited to one of carbocysteine, erdosteine, N-acetylcysteine, or a mixture thereof.
370. Any of the previous inventive concepts wherein the liquid 120 is comprising a anti-inflammatory medication, including but not limited to Methylxanthines.
371. Any of the previous inventive concepts wherein the liquid 120 is comprising nicotine.
372. Any of the previous inventive concepts wherein the liquid 120 is comprising an antimicrobial agent, including but not limited to one of Silver nanoparticles, PVP Iodine, tobramycin, colistin, and aztreonam lysine.
373. Any of the previous inventive concepts wherein the liquid 120 is comprising a vaccine.
374. The previous inventive concept wherein the vaccine is a vaccine for a respiratory infection caused by a virus selected from influenza or corona viruses.
375. Any of the previous inventive concepts wherein the liquid 120 is comprising a cannabinoid substance.
376. Any of the previous inventive concepts wherein the liquid 120 is comprising a an aqueous emulsion.
377. The previous inventive concept wherein the emulsion comprises droplets having a size distribution peak at droplets size larger than 5 nm and smaller than 100 nm.
378. The previous inventive concept wherein the emulsion comprises droplets having a size distribution peak at droplets size larger than 10 nm and smaller than 90 nm.
379. The previous inventive concept wherein the emulsion comprises droplets having a size distribution peak at droplets size larger than 20 nm and smaller than 80 nm.
380. Any of the previous inventive concepts wherein the emulsion liquid has surface tension such that the contact angle of a 3 mm diameter droplet of the liquid with mesh membrane is less than 90 degrees.
381. Any of the previous inventive concepts wherein the liquid 120 is comprising an aqueous colloid of nanoparticles, wherein the nanoparticles have a zeta potential of size greater than 10 mV.
382. The previous inventive concept wherein the nanoparticles diameter is having a size distribution peak at size larger than 2 nm and smaller than 100 nm.
600. An inhalation delivery system for carrying out a drug inhalation procedure by a human user comprising:
   a. an aerosol delivery module comprising a piezo assembly including: (i) an ultrasonically vibrable mesh membrane, (ii) a distal fluids compartment proximal to the mesh membrane in fluid communication with the mesh membrane, and (iii) an aerosol outlet for a mist comprising droplets of the liquid and generated by the mesh membrane; (iv) a distally extended mouthpiece in fluid communication with the mesh membrane and the aerosol outlet, the distal end of the system being the distal end of the mouthpiece;
   b. a control module comprising (i) a battery power source, (ii) a control circuitry for electrically powering the piezo assembly, (iii) an inhalation sensor, (iv) an activation switch configured for switching the control module between an initial OFF-state to a subsequent ON-state; and
   c. a liquid comprising a drug formulation;
   wherein when the inhalation system is in an assembled state such that the aerosol delivery module is attached to the control module and the liquid is contained within the distal fluids compartment in fluid communication with the mesh membrane, the system is configured to have at least two activation states such that,
   when the system is at an OFF-state the control circuitry does not drain power from the battery;
   wherein the control circuitry is configured to detect and/or distinguish between at least two inhalation states, (A) a non-inhalation state, and (B) an inhalation event state; and
   when the activation switch is at an ON-state, the control circuitry is pre-set such that (i) the piezo assembly is powered to vibrate the mesh membrane and emit a mist of the liquid only after the detecting an inhalation event conditioned on a sensor threshold limit (ii) a pre-determined first dose quantity of the liquid is configured for delivery, the first dose quantity is more than 0.1 mL and less than 2 mL.
601. Inventive concept 600 wherein a distal fluids compartment proximal to the mesh membrane is in fluid communication with the mesh membrane.
602. Inventive concept 600 wherein the system further comprises an indicator module comprising an indicator switchable between an Indicator-OFF state and an Indicator-ON state.
603. The previous inventive concept wherein the indicator is a light indicator.
604. The previous inventive concept wherein the light indicator is an LED.
605. Any of the previous 3 inventive concepts wherein, when the indicator is in an Indicator-ON state, the indicator light is switchable between two or more colors illumination states.
606. Any of the previous 4 inventive concepts wherein the indicator also emits a sound signal.
607. The previous inventive concept wherein the indicator is switchable between two or more sound emitting states distinguished by sound pitch and/or sound rhythm or pulsation.
608. Any of the previous 6 inventive concepts wherein the indicator state is automatically switching such that the indicator state when the system is at an OFF-state is different from the indicator state when the system is at an ON-state.
609. Any of the previous 7 inventive concepts wherein there is a correspondence between the indicator state and the system state.
610. The previous inventive concept wherein the indicator state is externally visible and/or audible, thereby providing an externally communicated indication of the state of the system.
611. Inventive concept 600, or any of the previous inventive concepts, wherein, when the system is at an ON-state, the system is switchable between at least two activation states selected from (A) an Idle-ON-state at which power is communicated from the battery to the control circuitry but the mesh membrane does not emit a mist, and (B) an active-ON-state at which the piezo assembly is powered to vibrate the mesh membrane and emit a mist of the liquid only after the inhalation sensor detects an inhalation event.
612. The previous inventive concept wherein the switching between the at least two activation states is conditioned on the control circuitry detection of an inhalation event.
613. Any of the previous 2 inventive concepts wherein when the system is at an ON-state, the piezo assembly is powered to vibrate the mesh membrane and emit a mist of the liquid for not more than 2 seconds after the inhalation sensor stops detecting an inhalation event.
614. The previous inventive concept wherein when the system is at an ON-state, the piezo assembly is powered to vibrate the mesh membrane and emit a mist of the liquid for not more than 1 second after the inhalation sensor stops detecting an inhalation event.
615. Inventive concept 600 wherein, when the system is at an ON-state, the piezo assembly is powered to vibrate the mesh membrane and emit a mist of the liquid only after and during the inhalation sensor detects an inhalation event.
616. Inventive concept 600 wherein, when the system is at an ON-state, the piezo assembly is powered to vibrate the mesh membrane and emit a mist of the liquid only after the inhalation sensor detects an inhalation event and for a pre-set duration of more than 0.1 second and less than 5 seconds.
617. The previous inventive concept wherein the piezo assembly is powered to vibrate the mesh membrane and emit a mist of the liquid only after the inhalation sensor detects an inhalation event and for a pre-set duration of more than 1 second and less than 3 seconds.
618. Inventive concept 600 wherein the inhalation sensor is a flow sensor configured to measure the air flow created by a human user one of at least inhaling and/or exhaling air.
619. The previous inventive concept wherein the inhalation event is detected as a flow of air.
620. The previous inventive concept wherein the sensor threshold limit is a minimal flow rate.
621. Inventive concept 600 wherein the inhalation sensor is a pressure sensor.
622. The previous inventive concept, wherein when the inhalation sensor is in fluid communication with a port displaced distally therefrom, the inhalation sensor being effective to detect an air pressure difference between an air pressure at the port and an ambient air pressure proximal to the port.
623. The previous inventive concept wherein the sensor threshold limit is a minimal pressure difference.
624. The previous inventive concept wherein, when in use, the port is located more distal than the proximal most location of engagement of the lips of the user with the system casing.
625. Any of the previous 3 inventive concepts wherein the inhalation sensor is effective to detect a difference between an air pressure in the inhalation flow-path and an ambient air pressure outside the inhalation device.
626. Any of the previous 4 inventive concepts, comprising control circuitry configured to initiate and/or cease activation of the piezo assembly in response to detection of an air pressure difference greater than a threshold limit, and/or cease activation of the piezo assembly in response to detection of an air pressure difference less than a pressure threshold limit.
627. The inhalation system of inventive concept 30, wherein the threshold limit is a pressure more than 1 cm H₂O and less than 10 cm H₂O.
628. The previous inventive concept wherein the threshold limit is a pressure less than 5 cm H2O.
629. The previous inventive concept wherein the threshold limit is a pressure more than 2 cm H2O.
630. The inhalation system of any one of the previous 8 inventive concepts, comprising a lumen for establishing fluid communication between the port and the inhalation sensor, the lumen including a first lumen portion in the control module and a second lumen portion in the aerosol delivery module.
631. Any of the previous 12 inventive concepts wherein when and/or after an inhalation event is not detected the system is in or switching into a non-inhalation state.
632. Any of the previous 13 inventive concepts wherein a non-inhalation detection is when the inhalation sensor and/or the control module do not detect an inhalation event.
633. Any of the previous 14 inventive concepts wherein a non-inhalation state is a state at which and/or after the control circuitry and/or the inhalation sensor do not detect an inhalation event.
634. Inventive concept 600 wherein the system further comprises a fill-level sensor comprising at least two electrodes exposed to interior of the liquid chamber and electronically connected to control circuitry, wherein the fill level sensor measures the resistance between the two electrodes.
635. The previous inventive concept wherein a detection of a change in the liquid fill-level of the fluids compartment corresponds to a change in the sensor resistance measurement and/or to an increase of the resistance measurement above a limit value.
636. Any of the previous 2 inventive concepts wherein a pre-determined fill-level sensing state or limit value by the sensor and control circuitry corresponds to an End-of-dose event detection.
637. Any of the previous 3 inventive concepts wherein one or more of the electrodes of the fill level sensor is located near the bottom of the distal liquids chamber.
638. The previous inventive concept wherein an increase resistance corresponds to a low-fill state.
639. Any of the previous inventive concepts wherein the fluids cartridge device further comprises a fill-level sensor, in the form of at least one or more electrodes, the fluids cartridge device comprising externally exposed fill-sensor-contacts connected to the at least one electrode of the fill sensor.
640. The previous inventive concept wherein, when in assembled state with the fluids cartridge device inserted, the fill-sensor contacts are in electronic communication with the electronic circuitry of the control module.
641. Any of the previous 7 inventive concepts wherein an End-of-dose state is further characterized by a low-fill detection.
642. Inventive concept 600 wherein a longitudinal axis of the mouthpiece is perpendicular to the plane tangential to the center of the mesh membrane.
643. The previous inventive concept wherein the longitudinal axis of the mouthpiece defines the longitudinal axis of the device system in the assembled state.
644. Inventive concept 600 wherein, when is use by a human, the lips and/or the teeth of the user engage with the mouthpiece such that the longitudinal axis of the mouthpiece is oriented distally towards the inside of the mouth.
645. Inventive concept 600 wherein the mesh membrane is located proximally to the middle of the mouthpiece.
646. Inventive concept 600 wherein the mesh membrane is located distally to the middle of the mouthpiece.
647. Inventive concept 600 wherein the mesh membrane is connected to and housed within the distal 50% of the mouthpiece.
648. Any of the previous 6 inventive concepts, wherein both (i) the piezo assembly comprising the mesh membrane, and (ii) the majority of the fluids compartment is housed within the mouthpiece.
649. The previous inventive concept wherein, when is use by a human, the lips and/or the teeth of the user engage with the mouthpiece such that both (i) the piezo assembly comprising the mesh membrane, and (ii) the majority of the fluids compartment is within the oral cavity of the human user.
650. Any of the previous 8 inventive concepts wherein the mouthpiece further comprises a proximal wide section, the proximal wide section having a cross section width that is greater than minimal width of the narrow section.
651. Any of the previous 6 inventive concepts wherein the mouthpiece comprises of a narrow section characterized by the following: (i) a minimum cross-sectional dimension of the narrow section is at least 10% smaller than a minimum cross-sectional dimension of the aerosol delivery module at the mesh membrane.
652. The previous inventive concept wherein a narrow section characterized by the following: (ii) when the user's lips and/or teeth are transversely engaged with the narrow section, the mesh membrane resides within the user's oral cavity and the aerosol outlet is in direct fluid communication with the user's oropharynx. 653.
654. Inventive concept 600 wherein the liquid is a drug formulation containing Epinephrine at a concentration greater than 0.5 mg/mL and less than 8 mg/mL.
655. The previous inventive concept wherein the liquid is a drug formulation containing Epinephrine at a concentration greater than 1 mg/mL.
656. The previous inventive concept wherein the liquid is a drug formulation containing Epinephrine at a concentration greater than 2 mg/mL.
657. The previous inventive concept wherein the liquid is a drug formulation containing Epinephrine at a concentration less than 5 mg/mL.
658. The previous inventive concept wherein the liquid is a drug formulation containing Epinephrine at a concentration is about 4 mg/mL.
659. Inventive concept 600 wherein, the dose quantity of the liquid is configured for delivery such that the dose is more than 0.2 mL.
660. The previous inventive concept wherein the dose is more than 0.4 mL.
661. The previous inventive concept wherein the dose is more than 0.6 mL.
662. The previous inventive concept wherein the dose is more than 0.8 mL.
663. The previous inventive concept wherein the dose is less than 1.2 mL
664. Any of the previous inventive concepts wherein, the dose quantity of the liquid is configured for delivery such that the total amount of Epinephrine contained in the first dose quantity is more than 0.3 mg and less than 8 mg.
665. The previous inventive concept wherein the total amount of Epinephrine contained in the first dose quantity is more than 0.5 mg.
666. The previous inventive concept wherein the total amount of Epinephrine contained in the first dose quantity is more than 1 mg.
667. The previous inventive concept wherein the total amount of Epinephrine contained in the first dose quantity is less than 6 mg.
668. The previous inventive concept wherein the total amount of Epinephrine contained in the first dose quantity is less than 4 mg.
669. The previous inventive concept wherein the total amount of Epinephrine contained in the first dose quantity is about 2 mg.
670. Inventive concept 600 wherein, the dose quantity of the liquid is configured for delivery such that the dose quantity is predetermined by a corresponding dose-total-activation-period, the dose-total-activation-period being a combined activation time during which the piezo assembly is powered to vibrate the mesh membrane and emit a mist of the liquid.
671. The previous inventive concept wherein the system and/or the control module automatically switch to the OFF-state after the combined activation time during which the piezo assembly is powered to vibrate the mesh membrane and emit a mist of the liquid is equal or greater than a pre-set dose-total-activation-period.
672. Inventive concept 600 further comprising an End-of-dose state characterized and/or detected by one of (i) detecting that the dose quantity has been delivered, and (ii) the indicator is changing its activation state.
673. The previous inventive concept wherein detecting that the dose quantity has been delivered is determined by at least one of: (i) the quantity of liquid in the fluids compartment is reduce from a previous amount by an amount equal to about the predetermined dose quantity, (ii) a corresponding dose-total-activation-period has been completed, (iii) a pre-determined fill-level sensing state or limit value by the sensor and control circuitry.
674. Any of the previous 4 inventive concepts wherein an indicator is changing its activation state.
675. The previous inventive concept wherein the indicator light is switching from one color state to another color.
676. Any of the previous two inventive concepts wherein the indicator is switching from one sound emitting state to another.
677. Inventive concepts 600, 601, any of the previous 7 inventive concepts wherein at least a portion of the distal fluids compartment bounding walls is of sufficient transparency such that a level of the liquid fill level surface is visually discernable as a change of color or hue.
678. Inventive concept 600 wherein the fluids compartment is sized sufficiently to contain both a first dose quantity and a second dose quantity of the same size as the first dose quantity.
679. The previous inventive concept wherein the control circuitry is configured such that a switch needs to be engaged to enable the administration of a second or subsequent dose if needed by a user.
680. Any of the previous two inventive concepts wherein an indicator is pre-set to indicate the termination of the second dose quantity.
681. Inventive concept 600 wherein the system and the control module configured to be is initially at an OFF-state.
682. Any of the previous inventive concepts wherein the drug inhalation delivery system is pre-assembled.
683. Inventive concept 600 wherein a default pre-set is determined for the values of (i) the sensor threshold limit, (ii) the dose quantity.
684. Inventive concept 600 wherein at least a portion of the liquid is within the distal fluids compartment.
685. Inventive concept 600 wherein the liquid comprising a drug formulation is pre-filled within the distal fluids compartment.
686. Inventive concept 600 wherein the liquid comprising a drug formulation is pre-filled and sealed within the distal fluids compartment.
687. Inventive concept 600 wherein the liquid is sterile.
688. Inventive concept 600 wherein the system is further comprising a distal cap.
689. The previous inventive concept wherein the distal cap is a protective cover.
690. The previous two inventive concepts wherein the distal cap is connected to the mouthpiece.
691. Any of the previous 2 inventive concepts wherein the cap is a portion of the mouthpiece.
692. The previous inventive concept wherein the cap is a removable portion of the mouthpiece.
693. The previous inventive concept wherein the cap is reversibly attachable to the mouthpiece.
694. Any of the previous 5 inventive concepts wherein the distal cap is at least partially covering over the aerosol outlet.
695. Any of the previous 6 inventive concepts wherein the cap is removable.
696. Any of the previous 7 inventive concepts wherein the cap is a switch such that a movement of the cap enable switching the control module between an initial OFF-state to a subsequent ON-state.
697. The previous inventive concept wherein the cap is an activation switch such that a movement of the cap is switching the control module between an initial OFF-state to a subsequent ON-state.
698. Any of the previous 2 inventive concepts wherein a movement of the cap is a removal of the cap.
699. Any of the previous 3 inventive concepts wherein a movement of the cap is a turning of the cap.
700. Any of the previous 4 inventive concepts wherein the cap is a reversible switch such that reversing a movement of the cap enable switching the control module between an initial ON-state to a subsequent OFF-state.
701. Any of the previous 5 inventive concepts wherein the movement of the cap closes an electric contact or an electric circuit which is previously open.
702. Inventive concept 600 wherein the activation switch is a reversible switch such that reversing the configuration of the switch enable switching the control module between an initial ON-state to a subsequent OFF-state.
703. Inventive concept 600 wherein the activation switch is non-reversible such that the switching the control module between an initial ON-state to a subsequent OFF-state is blocked.
704. Any of the previous two inventive concepts wherein the switch element is selected from: a cap, a press button, a touch contact, a lever movement.
705. Any of the previous 3 inventive concepts wherein a first engagement with the switch is switching the system and/or the control module from an OFF-state to an ON-state.
706. The previous inventive concept wherein a second engagement with the switch is switching the system and/or the control module from an ON-state to an OFF-state.
707. The inventive concept 600 further comprising a rigid protective casing, the protective casing covers over the majority of the mouthpiece and the control module.
708. The previous inventive concept wherein the activation switch remains exposed for external user access without removal of the protective casing.
709. The previous inventive concept wherein in addition the distal end of the protective casing comprises a central aperture such that a mist can exit distally from the aerosol outlet without removal of the protective casing.
710. The previous inventive concept wherein a distal cap can be removed from the protective casing and/or the mouthpiece without removal of the protective casing.
711. Any of the previous inventive concepts wherein, when in use, the user lips and/or teeth engage with the protective casing covering over the mouthpiece.
712. Any of the previous inventive concepts wherein an elastic band is wrapped around at least a portion of the aerosol delivery module 630.
713. The previous inventive concept wherein the portion is a distal section of the aerosol delivery module 630.
714. Any of the previous 2 inventive concepts wherein the elastic band is reversibly removable
715. Any of the previous 3 inventive concepts wherein the elastic band also covers over an inlet or a filling port 103 into the liquids chamber.
716. Any of the previous 4 inventive concepts wherein the elastic band is further comprising a taste-producing surface section.
717. The previous inventive concept wherein the taste-producing surface is comprising a flavoring agent and/or a scented embedded agent and/or coating.
718. Any of the previous inventive concepts wherein the system is comprising, in addition to the filling port 103 into the fluids chamber 105, also a secondary port 113 of smaller cross section than the filling port 103.
719. The previous inventive concept wherein, port 113 is serving as an air outlet, for release of air from the fluids chamber, simultaneously with liquids filling via the filling port 103. In preferred embodiments, the cross section area of the filling port 103 is smaller than 1 cm² and bigger than 0.1 cm², or smaller than 0.5 cm². In some preferred embodiments, the cross-section area of the secondary filling port 113 is smaller than 0.3 cm² and bigger than 0.001 cm², or smaller than 0.1 cm².
720. The system of inventive concepts 600 or 601 wherein the aerosol delivery module further comprising a deflecting protrusion within the distal fluids compartment, wherein the deflecting protrusion extends from a protrusion-distal-end located in proximity to the mesh membrane to a protrusion-proximal-end located at least 2 mm more proximal from the mesh membrane than the protrusion-distal-end.
721. The previous inventive concept wherein the deflecting protrusion is raised from the floor of the distal fluids compartment by more than 1 mm and less than 10 mm.
722. The previous inventive concept wherein the deflecting protrusion is effective to deflect air bubbles preferentially upwards and/or sideways away from the mesh membrane for air bubbles coming into the distal liquids chamber through the mesh membrane.
723.
724.
800. A method for performing a drug dose inhalation using an inhalation system, wherein, the inhalation system is comprising:
   a. an aerosol delivery module comprising: a piezo assembly including an ultrasonically vibrable mesh membrane, a distal fluids compartment in fluid communication with the mesh membrane, an aerosol outlet; and a distally extended mouthpiece in fluid communication with the mesh membrane and the aerosol outlet;
   b. a control module comprising (i) a battery power source, (ii) a control circuitry for electrically powering the piezo assembly, (iii) an inhalation sensor, (iv) an activation switch configured for switching the control module between an initial OFF-state to a subsequent ON-state; and
   c. a liquid comprising a drug formulation contained within the fluids compartment;
wherein the control circuitry is configured to detect and/or distinguish between at least two inhalation states, (A) a non-inhalation state, and (B) an inhalation event state; and
wherein the method comprising:
   i. the system is initially at an OFF-state at which the control circuitry does not drain power from the battery;
   ii. engaging the activation switch thereby switching the system from an OFF-state to and ON-state at which power is communicated from the battery to the control module;
   iii. placing the distal tip of the mouthpiece inside the mouth such that the lips engage an outer surface of the mouthpiece and/or a protective covering over the mouthpiece;
   iv. detecting an inhalation event conditioned on a sensor threshold limit;
   v. powering the piezo assembly to vibrate the mesh membrane and emit a mist of the liquid only after detecting an inhalation event;
   vi. automatically subsiding and/or stopping the powering of the piezo assembly to subside or stop the emission of a mist after detecting a non-inhalation state;
   vii. Indicating that a pre-determined first dose quantity of the liquid has been delivered, the first dose quantity is more than 0.1 mL and less than 2 mL.
801. The previous inventive concept wherein the inhalation system is characterized by any of the inventive concepts 600 to 722.
802. Any of the inventive concepts 800 or 801, further comprising the step of turning on an external visible and/or audible indicator in parallel with at least a portion of the time when powering the piezo assembly to vibrate the mesh membrane.
803. Any of the inventive concepts 800 or 801, further comprising the step of detecting that a dose quantity has been delivered.
804. Any of the inventive concepts 800 or 801, further comprising the step of detecting an End-of-dose state.
805. Any of the previous 3 inventive concepts further comprising the step changing the activation state of an of indicator after detecting that a dose quantity has been delivered.
806. Any of the previous 6 inventive concepts wherein, the placing the distal tip of the mouthpiece inside the mouth is positioned such that both the mesh membrane and the majority of the distal fluids compartment volume is within the oral cavity distally from the lips.
807. Any of the previous 7 inventive concepts wherein the placing the distal tip of the mouthpiece inside the mouth is positioned such that the lips and/or the teeth engage in contact with the narrow section.
808. Any of the inventive concepts 800 or 801 wherein the switching the system from an OFF-state to and ON-state comprise of moving a distal cap or protective cover.
809. The previous inventive concept wherein the moving of the distal cap or protective cover comprises of removing the distal cap or protective cover.
810. Any of the previous 10 inventive concepts wherein the switching the system from an OFF-state to and ON-state is simultaneously also setting the system into a default state of powering the piezo assembly to vibrate the mesh membrane and emit a mist of the liquid only after detecting an inhalation event.
811. The previous inventive concept wherein the switching the system from an OFF-state to and ON-state is simultaneously also setting the system into a default state with a predetermine dose quantity.
812. The previous inventive concept wherein the switching the system from an OFF-state to and ON-state is simultaneously also setting the a predetermine End-of-dose state.
850. Any of the inventive concepts 1 or 2 or 253 or 302 or 313 or 324 or 332 or 349 or 352 or 600 or 800, wherein the aerosol delivery module is further comprising a bottom-air-channel or air conduit element 527, the bottom-air channel is passing along a path that is passing around a side of the mesh membrane from a more proximal to a more distal side of the mesh membrane, at a level at least partially or predominantly below the center the mesh membrane.
851. The previous inventive concept wherein the bottom-air-channel element 527 is comprising a proximal entry port 528 situated proximally to the mesh membrane, and a distal exit port 529 situated distally to the mesh membrane at a level below the center the mesh membrane.
852. The previous inventive concept wherein, when in assembled state the aerosol delivery module is positioned in use inside the mouth of a human user, the proximal entry port 528 is situated outside of the mouth and enables ambient entering-air-flow 525 to enter into the bottom-air-channel element 527, and to a distal exiting-air-flow 526 via a distal exit port 529 at a level below the center the mesh membrane inside the mouth.
853. The previous inventive concept wherein there is reduced portion the aerosol ejected 141 from the mesh membrane that collide with and settle onto the tongue tissue, compared when no air flows in the bottom-air-channel element 527.
854. Any of the inventive concepts 1 or 2 or 253 or 302 or 313 or 324 or 332 or 349 or 352 or 600 or 800 or 850, wherein the aerosol delivery module 630 further comprising a tongue-depressing element 688 which extends more distally from the mesh membrane 185 by at least 5 mm and preferably less than 30 mm.
855. The previous inventive concept wherein the tongue-depressing element 688 extends at a level at least 3 mm, or at least 5 mm, below the center of mesh membrane.
856. The previous inventive concept wherein the tongue-depressing element 688 extends at a level below the edge of mesh membrane.
857. Any of the previous 3 inventive concepts wherein at least a portion of the tongue-depressing element 688 extends at a at level below the bottom-air-channel element 527.
900. Any of the previous inventive concepts wherein the liquid is comprising a pharmaceutical active substance.
901. The previous inventive concepts wherein the liquid is comprising an FDA approved drug.
902. Any of the previous 2 inventive concepts wherein the liquid is comprising a bronchodilator medication, including but not limited to one of albuterol, levalbuterol, ipratropium, aclidinium, arformoterol, formoterol, glycopyrrolate, indacaterol, olodaterol, revefenacin, salmeterol, tiotropium, umeclidinium, Terbutaline, or a mixture thereof.
903. Any of the previous 3 inventive concepts wherein the liquid is comprising a corticosteroid medication, including but not limited to one of Fluticasone, Budesonide, Prednisolone, or a mixture thereof.
904. Any of the previous 4 inventive concepts wherein the liquid is comprising a muscarinic medication, including but not limited to one of Revefenacin, Ipratropium bromide, Tiotropium bromide, or a mixture thereof.
905. Any of the previous 5 inventive concepts wherein the liquid is comprising a Mucoactive medication, including but not limited to one of carbocysteine, erdosteine, N-acetylcysteine, or a mixture thereof.
906. Any of the previous 6 inventive concepts wherein the liquid is comprising a anti-inflammatory medication, including but not limited to Methylxanthines.
907. Any of the previous 7 inventive concepts wherein the liquid is comprising nicotine.
908. Any of the previous 8 inventive concepts wherein the liquid is comprising an antimicrobial agent, including but not limited to one of Silver nanoparticles, PVP Iodine, tobramycin, colistin, and aztreonam lysine.
909. Any of the previous 9 inventive concepts wherein the liquid is comprising a vaccine.
910. The previous inventive concept wherein the vaccine is a vaccine for a respiratory infection caused by a virus selected from influenza or corona viruses.
911. Any of the previous 11 inventive concepts wherein the liquid is comprising a cannabinoid substance.
912. Any of the previous 12 inventive concepts wherein the liquid comprises a B12 vitamin.
913. Any of the previous 13 inventive concepts wherein the liquid is comprising an aqueous emulsion of droplets encapsulating the drug or active substance.
914. Any of the previous 14 inventive concepts wherein the liquid is comprising encapsulating nanoparticles dispersed within an aqueous solution.
915. Any of the previous 2 inventive concepts wherein the nanoparticles or droplets having a size distribution peak at droplets size larger than 5 nm and smaller than 1000 nm.
916. Any of the previous 3 inventive concepts wherein the nanoparticles or droplets diameter is having a size distribution peak at size larger than 600 nm and smaller than 900 nm.
917. Any of the previous 4 inventive concepts wherein the nanoparticles diameter is having a size distribution peak at size larger than 300 nm and smaller than 600 nm.
918. Any of the previous 5 inventive concepts wherein the nanoparticles diameter is having a size distribution peak at size larger than 100 nm and smaller than 300 nm.
919. Any of the previous 6 inventive concepts wherein the nanoparticles diameter is having a size distribution peak at size larger than 50 nm and smaller than 100 nm.
920. Any of the previous 6 inventive concepts wherein the nanoparticles diameter is having a size distribution peak at size larger than 10 nm and smaller than 50 nm.
921. Any of the previous inventive concepts wherein the emulsion liquid has surface tension such that the contact angle of a 3 mm diameter droplet of the liquid with mesh membrane is less than 90 degrees.
922. Any of the previous inventive concepts wherein the liquid is comprising an aqueous colloid of nanoparticles, wherein the nanoparticles have a zeta potential of size greater than 10 mV.
923. The previous inventive concept wherein the nanoparticles diameter is having a size distribution peak at size larger than 2 nm and smaller than 100 nm.
924. Inventive concept 900 wherein the liquid further comprises a coloring agent.
925. Inventive concept 900 wherein the liquid includes a pH buffer stabilizing the liquid at a pH higher than pH=4 and lower than pH=8.
926. The previous inventive concept wherein the pH is less than pH=7.
927. The previous inventive concept wherein the pH is more than pH=6.
928. Inventive concept 900 wherein the liquid is further comprising a sweetening flavoring agent.
929. The previous inventive concept wherein the liquid surface tension is between 70 mN/m to 75 mN/m at 25 degrees Celsius.
930. The previous inventive concept wherein the sweetening agent is sugar.
931. Inventive concept 900 wherein the liquid is further comprising a salting flavoring agent.
932. The previous inventive concept wherein the liquid surface tension is between 70 mN/m to 75 mN/m at 25 degrees Celsius.
933. The previous inventive concept wherein the salting agent is comprising sodium chloride.

### CONCLUDING REMARKS

The invention is herein described, by way of example only, with reference to the accompanying drawings. With specific reference now to the drawings in detail, it is stressed that the particulars shown are by way of example and for purposes of illustrative discussion of the embodiments of the exemplary system only and are presented in the cause of providing what is believed to be a useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice and how to make and use the embodiments.

For brevity, some explicit combinations of various features are not explicitly illustrated in the figures and/or described. It is now disclosed that any combination of the method or device features disclosed herein can be combined in any manner—including any combination of features—any combination of features can be included in any embodiment and/or omitted from any embodiments.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

It will be clear to the skilled artisan that any of the features described in connection with any of the figures can be combined with each other with the scope of the present invention even if not explicitly combined in this disclosure. For example, a design for which an airflow sensor was not explicitly shown may include an airflow sensor to trigger activation/initiation (or deactivation/cessation) of the piezo assembly and generation of mist by the mesh membrane, or a design for which a capillary pathway was not explicitly shown may include a capillary path for transport of liquid to the mesh membrane. As another non-limiting example, any of the designs illustrated can incorporate a liquid-retaining compartment effective to be filled using gravity and to retain liquid using a liquid-retaining wall, such that the mesh remains in contact with liquid, after the inhalation device is turned horizontal or ‘below horizontal’, i.e., with the container at least partly higher than the liquid-retaining compartment.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. An inhalation system for delivery of an aerosol to the oropharynx of a human user, the inhalation system comprising:
a. an aerosol delivery module comprising a piezo assembly including: an ultrasonically vibrable mesh membrane, a distal fluids compartment in fluid communication with the mesh membrane, and an aerosol outlet for a mist comprising droplets of the liquid generated by the mesh membrane; and
b. a control module,
wherein when the inhalation system is in an assembled state such that the aerosol delivery module is distally disposed and/or the control module is attached thereto, the assembled inhalation system is shaped to include a narrow section characterized by the following:
a minimum cross-sectional dimension of the narrow section is at least 10% smaller than a minimum cross-sectional dimension of the aerosol delivery module at the mesh membrane, and/or
when the user's lips and/or teeth are transversely engaged with the narrow section, the mesh membrane resides within the user's oral cavity and the aerosol outlet is in direct fluid communication with the user's oropharynx.

2. An inhalation system for delivery of an aerosol to the oropharynx of a human user, the inhalation system comprising:
a. an aerosol delivery module comprising a piezo assembly including: an ultrasonically vibrable mesh membrane, a distal fluids compartment in fluid communication with the mesh membrane, and an aerosol outlet for a mist comprising droplets of the liquid generated by the mesh membrane;
b. a control module;
c. a bottom-air-channel or air conduit element 527, the bottom-air channel is passing along a path that is passing around a side of the mesh membrane from a more proximal to a more distal side of the mesh membrane, at a level at least partially or predominantly below the center the mesh membrane.
Wherein, when the inhalation system is in an assembled state, the aerosol delivery module is attached to and/or is predominantly distally disposed thereto the control module.

3-290. (canceled)

291-317. (canceled)

318. (canceled)

319. (canceled)

320. (canceled)

321. (canceled)

322. (canceled)

323. (canceled)
